(19) United States
(12) Patent Application Publication
(10) Pub. No.: US 2022/0047196 A1
(43) Pub. Date: Feb. 17, 2022

(54) MOTION SICKNESS STATE DETERMINATION SYSTEM, BIOLOGICAL INFORMATION ACQUISITION DEVICE, SURROUNDING ENVIRONMENT INFORMATION ACQUISITION DEVICE, MOTION SICKNESS STATE DETERMINATION DEVICE, AND MOTION SICKNESS STATE DETERMINATION METHOD

(71) Applicant: SONY SEMICONDUCTOR SOLUTIONS CORPORATION, KANAGAWA (JP)

(72) Inventor: NOBUYUKI KUBOI, TOKYO (JP)

(21) Appl. No.: 17/310,307

(22) PCT Filed: Dec. 19, 2019
(86) PCT No.: PCT/JP2019/049889

§ 371 Date: Jul. 27, 2021

(30) Foreign Application Priority Data
Feb. 5, 2019 (JP) 2019-018683

(51) Int. Cl. A61B 5/16; A61B 5/00; A61B 5/1486; G06F 3/01

(52) U.S. Cl. CPC A61B 5/165; A61B 5/4076; G06F 3/014; A61B 5/1486; A61B 5/4011

(57) ABSTRACT

An object of the present technology is to accurately determine a motion sickness state of an individual subject. The present technology provides a motion sickness state determination system (1) that includes a first index acquisition unit (12) that acquires biological information of a subject as a first motion sickness state index, a second index acquisition unit (13) that acquires a second motion sickness state index on the basis of movement information regarding a movement of the subject and/or surrounding environment information perceived by the subject, and a motion sickness state determination unit (14) that determines whether or not the subject will be in a motion sickness state or is in the motion sickness state on the basis of the first motion sickness state index and the second motion sickness state index. Furthermore, the present technology also provides a motion sickness state determination device (10), a biological information acquisition device (20), and a surrounding environment information acquisition device (30) included in the system, and a motion sickness state determination method.

### TECHNICAL FIELD

The present technology relates to a motion sickness state determination system, a biological information acquisition device, a surrounding environment information acquisition device, and a motion sickness state determination method. More specifically, the present technology relates to a motion sickness state determination system that determines a motion sickness state of a subject on the basis of two motion sickness state indexes, a biological information acquisition device, a surrounding environment information acquisition device, and a motion sickness state determination device included in the system, and a motion sickness state determination method for determining the motion sickness state of the subject on the basis of the two motion sickness state indexes.

### BACKGROUND ART

Many people suffer from motion sickness in vehicles such as automobiles, buses, ships, or airplanes in their daily lives, and there is a great need for countermeasures. Furthermore, in recent years, a motion sickness caused when a virtual reality (VR) game that has been rapidly widespread is continuously played has been a big issue that is discussed in academic conferences and International telecommunication Union (ITU).

As a countermeasure against the motion sickness, medicines mainly including antihistamine are sold generally. The functionality of the medicine has been improved, for example, a duration time of an effect of a single dose is lengthened or the medicine can be taken without water.

Some countermeasures have been proposed not only from the viewpoint of medicines but also from the viewpoint of devices. For example, Patent Document 1 discloses a vehicle occupant posture control device that prevents occurrence of a motion sickness of an occupant. The device includes a plurality of detection units that detects information around a vehicle and a state of each vehicle, a change unit that changes a supporting state of a support unit that supports the body of the occupant, and a driving control unit that generates a traveling plan on the basis of the detection result of the detection unit, controls driving of the vehicle according to the generated traveling plan, predicts accelerations in the vehicle front-back direction and the vehicle width direction generated in the vehicle after a predetermined time elapses from the current time on the basis of the generated traveling plan, starts to change the supporting state according to the predicted acceleration, and controls the change unit so as to set the supporting state according to the predicted acceleration before a predetermined time elapses.

Furthermore, Patent Document 2 discloses a wearable device that has a low frequency wave generation function for transmitting low frequency waves to the median nerve of the wrist so that nausea and vomiting due to the motion sickness, morning sickness, drugs, or the like can be effectively blocked. The device is worn on the wrist and includes a main body that includes a low frequency wave generation unit that generates the low frequency waves.

As countermeasures against VR sickness, countermeasures using visual effects are considered, for example, an acceleration effect in a game video is visualized, a field of view in a video is intentionally narrowed, and a line-of-sight direction of a user is clearly indicated in a video.

### CITATION LIST

### Patent Document

Patent Document 1: Japanese Patent Application Laid-Open No. 2017-71370
Patent Document 2: Japanese Patent Application Laid-Open No. 2018-511453

### SUMMARY OF THE INVENTION

### Problems to be Solved by the Invention

However, in a case where the medicine is used as the countermeasure against the motion sickness, a motion sickness suppressing effect may differ depending on the constitution of the user or the daily physical condition of the user, or for example, side effects such as drowsiness, or a feeling of malaise may occur. The medicine needs to be taken 30 minutes to one hour before a time when the effect is expected, and does not have immediacy. Moreover, many people are saying in actual that it is not desirable to make children take medicines.

Furthermore, the methods disclosed in Patent Documents 1 and 2 do not detect occurrence or a sign of a motion sickness state according to an individual user and are not able to effectively prevent the motion sickness.

Furthermore, the visual effect for VR sickness countermeasures is first created with a feeling of a game developer, and then, the created visual effect is finally adjusted on the basis of play questionnaires of the limited number of test users. Therefore, the motion sickness suppressing effect may differ for each user. Moreover, in anticipation of wider spread of the VR in the future, it is assumed that a wider variety of users play VR games. Therefore, it is easily estimated that it is more difficult to expect the effects.

As described above, it is required to accurately determine a motion sickness state of an individual user. Furthermore, it is required to effectively prevent or suppress the motion sickness state. Moreover, it is desirable to cope with the motion sickness state in real time, and in addition, it is desirable that the method for coping with the motion sickness state has no side effects and high immediacy.

An object of the present technology is to provide a new technique for solving at least one of the above problems.

### Solutions to Problems

The present inventors have found that the above problems can be solved by a motion sickness state determination system having a specific configuration.

That is, the present technology provides a motion sickness state determination system that includes a first index acquisition unit that acquires biological information of a subject as a first motion sickness state index, a second index acquisition unit that acquires a second motion sickness state index on the basis of movement information regarding a movement of the subject and/or surrounding environment information perceived by the subject, and a motion sickness state determination unit that determines whether or not the subject will be in a motion sickness state or is in the motion sickness state on the basis of the first motion sickness state index and the second motion sickness state index.

The motion sickness state determination system may further include a motion sickness state control unit that controls the motion sickness state of the subject on the basis of the determination result of the motion sickness state determination unit.

The motion sickness state control unit may determine processing content used to control the motion sickness state on the basis of information acquired from a database.

The motion sickness state determination system may determine the motion sickness state of the subject in real time.

In a case where both of the first motion sickness state index and the second motion sickness state index satisfy references set for the respective indexes, the motion sickness state determination unit may determine that the subject will be in the motion sickness state or is in the motion sickness state.

The biological information may include information regarding an enzyme derived from the subject.

The enzyme may be an amylase.

According to one embodiment of the present technology, the movement information is movement information regarding a movement of a body of the subject, the surrounding environment information is video information regarding a video that is visually perceived by the subject, and the second index acquisition unit may acquire a second motion sickness state index on the basis of the movement information and the video information.

In the embodiment, the movement information is first acceleration information based on the movement of the body of the subject, the surrounding environment information is second acceleration information based on the video visually perceived by the subject, and the second index acquisition unit may acquire a second motion sickness state index on the basis of the first acceleration information and the second acceleration information.

In the embodiment, the second index acquisition unit may acquire a difference between the first acceleration information and the second acceleration information as the second motion sickness state index.

In the embodiment, a motion sickness state control unit may be further included that applies a current to the head of the subject on the basis of the determination result of the motion sickness state determination unit.

According to another embodiment of the present technology, the surrounding environment information is odor information regarding an odor perceived by sense of smell by the subject, and the second index acquisition unit may acquire a second motion sickness state index on the basis of the odor information.

In the embodiment, the second index acquisition unit may acquire an intensity of a predetermined type of odor as the second motion sickness state index.

In the embodiment, a motion sickness state control unit may be further included that presents an odor to the subject or removes an odor around the subject on the basis of the determination result of the motion sickness state determination unit.

The motion sickness state determination system according to the present technology may further include an output unit that outputs the determination result of the motion sickness state determination unit.

Furthermore, the present technology provides a biological information acquisition device that includes a biological information acquisition unit that acquires biological information of a subject and is used in combination with an index acquisition device that acquires a motion sickness state index on the basis of movement information regarding a movement of the subject and/or surrounding environment information perceived by the subject in order to determine a motion sickness state of the subject.

Furthermore, the present technology provides a surrounding environment information acquisition device that includes a surrounding environment information acquisition unit that acquires surrounding environment information perceived by a subject and is used in combination with a biological information acquisition device that acquires biological information of the subject as a motion sickness state index in order to determine a motion sickness state of the subject.

Furthermore, the present technology provides a motion sickness state determination device that includes a first index acquisition unit that acquires biological information of a subject as a first motion sickness state index, a second index acquisition unit that acquires a second motion sickness state index on the basis of movement information regarding a movement of the subject and/or surrounding environment information perceived by the subject, and a motion sickness state determination unit that determines whether or not the subject will be in a motion sickness state or is in the motion sickness state on the basis of the first motion sickness state index and the second motion sickness state index.

Furthermore, the present technology provides a motion sickness state determination method that includes a first index acquisition process for acquiring biological information of a subject as a first motion sickness state index, a second index acquisition process for acquiring a second motion sickness state index on the basis of movement information regarding a movement of the subject and/or surrounding environment information perceived by the subject, and a motion sickness state determination process for determining whether or not the subject will be in a motion sickness state or is in the motion sickness state on the basis of the first motion sickness state index and the second motion sickness state index.

### BRIEF DESCRIPTION OF DRAWINGS

FIG. 1 is a diagram for explaining the principle of a motion sickness.

FIG. 2 is a schematic diagram for explaining mismatch between vestibular information and visual information.

FIG. 3 is a block diagram of an example of a motion sickness state determination system according to a first embodiment of the present technology.

FIG. 4A is a schematic diagram of an example of an enzyme sensor.

FIG. 4B is a block diagram of an example of the enzyme sensor.

FIG. 4C is a diagram illustrating a configuration example of the enzyme sensor.

FIG. 5 is a block diagram of a motion sickness state determination system of a first example according to the first embodiment.

FIG. 6 is a diagram illustrating a configuration example of the motion sickness state determination system of the first example according to the first embodiment.

FIG. 7 is an example of a flowchart of motion sickness state determination processing by the motion sickness state determination system of the first example according to the first embodiment.

FIG. 8 is a diagram illustrating an example of a fluctuation in an amylase activity.

FIG. 9 is a diagram illustrating another configuration example of the motion sickness state determination system of the first example according to the first embodiment.

FIG. 10 is a diagram for explaining a cloud database.

FIG. 11 is a diagram illustrating a current to be applied.

FIG. 12 is a block diagram of a motion sickness state determination system of a second example according to the first embodiment.

FIG. 13 is a diagram illustrating a configuration example of the motion sickness state determination system of the second example according to the first embodiment.

FIG. 14 is an example of a flowchart of motion sickness state determination processing by the motion sickness state determination system of the second example according to the first embodiment.

FIG. 15 is a block diagram of a motion sickness state determination system of a third example according to the first embodiment.

FIG. 16 is a diagram illustrating a configuration example of the motion sickness state determination system of the third example according to the first embodiment.

FIG. 17 is an example of a flowchart of motion sickness state determination processing by the motion sickness state determination system of the third example according to the first embodiment.

FIG. 18 is a block diagram of a motion sickness state determination system of a fourth example according to the first embodiment.

FIG. 19 is a diagram illustrating a configuration example of the motion sickness state determination system of the fourth example according to the first embodiment.

FIG. 20 is an example of a flowchart of motion sickness state determination processing by the motion sickness state determination system of the fourth example according to the first embodiment.

FIG. 21 is a block diagram of a motion sickness state determination system of a fifth example according to the first embodiment.

FIG. 22 is a diagram illustrating a configuration example of the motion sickness state determination system of the fifth example according to the first embodiment.

FIG. 23 is an example of a flowchart of motion sickness state determination processing by the motion sickness state determination system of the fifth example according to the first embodiment.

FIG. 24 is a block diagram of a motion sickness state determination system of a sixth example according to the first embodiment.

FIG. 25 is a diagram illustrating a configuration example of the motion sickness state determination system of the sixth example according to the first embodiment.

FIG. 26 is an example of a flowchart of motion sickness state determination processing by the motion sickness state determination system of the sixth example according to the first embodiment.

FIG. 27 is an example of a flowchart of a motion sickness state determination method according to the present technology.

FIG. 28 is a diagram illustrating a hardware configuration example of a motion sickness state determination device according to the present technology.

### MODE FOR CARRYING OUT THE INVENTION

Preferred embodiments for carrying out the present technology will be described below. Note that embodiments to be described below indicate representative embodiments of the present technology, and the scope of the present technology is not limited to only these embodiments. Note that description of the present technology will be made in the following order.

1. First Embodiment (motion sickness state determination system)

(1) Description of First Embodiment

(2) First Example of First Embodiment (example of determination based on amylase activity and acceleration)

(3) Second Example of First Embodiment (application example to VR devices)

(4) Third Example of First Embodiment (application example to VR game development)

(5) Fourth Example of First Embodiment (application example to attractions in amusement parks)

(6) Fifth Example of First Embodiment (application example to training simulators)

(7) Sixth Example of First Embodiment (example of determination based on amylase activity and odor)

2. Second Embodiment (biological information acquisition device)

3. Third Embodiment (surrounding environment information acquisition device)

4. Fourth Embodiment (motion sickness state determination device)

5. Fifth Embodiment (motion sickness state determination method)

### 1. First Embodiment (Motion Sickness State Determination System)

(1) Description of First Embodiment

In a motion sickness state determination system according to the present technology, it is determined whether or not a subject will be in a motion sickness state or is in the motion sickness state on the basis of a first motion sickness state index that is biological information of the subject and a second motion sickness state index acquired on the basis of movement information regarding a movement of the subject and/or surrounding environment information perceived by the subject. By determining the motion sickness state of the subject on the basis of the two indexes, it is possible to appropriately determine the motion sickness state of the subject. Moreover, because the motion sickness state determination system according to the present technology can appropriately determine the motion sickness state of the subject, it is possible to appropriately prevent or suppress the motion sickness state.

Hereinafter, the principle of a motion sickness will be described first, and then, a first embodiment according to the present technology will be described in detail.

(1-1) Principle of Motion Sickness

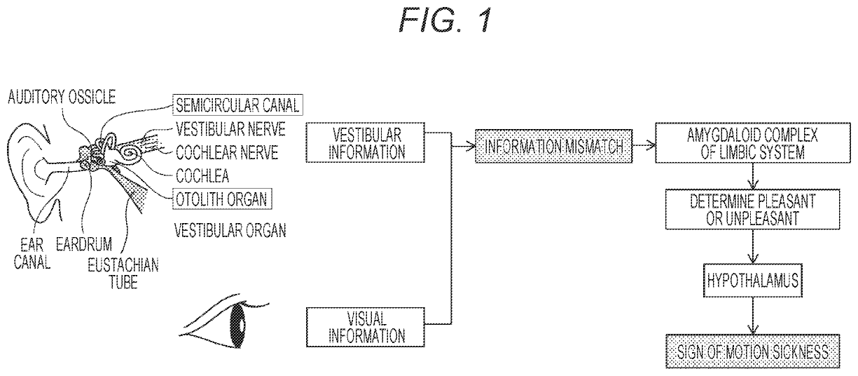

FIG. 1 is a diagram for explaining the principle of a motion sickness. As illustrated in FIG. 1, the vestibular organ is in the inner ear. The vestibular organ is an acceleration detection organ of a human body, and can sense a linear movement and a rotational acceleration. The vestibular organ includes the semicircular canal and the otolith organ. The semicircular canal senses motions in all of three-dimensional directions (rotational acceleration), and the otolith organ senses motions in the straight direction (linear acceleration) and the gravity. Furthermore, as illustrated in FIG. 1, humans visually acquire visual information, for example, external landscapes or the like.

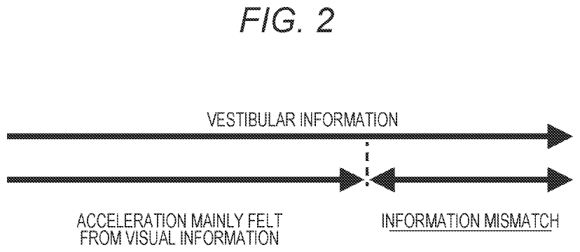

There is a case where the information sensed by the vestibular organ and the visual information visually perceived mismatch, for example, in a case where the subject is moving on a vehicle and in a case where the subject is watching a video with a VR device. A schematic diagram for explaining the mismatch between the vestibular information and the visual information is illustrated in FIG. 2. As illustrated in FIG. 2, the mismatch may occur, for example, in a case where the acceleration sensed by the vestibular organ is larger than the acceleration based on the visual information visually perceived. The reverse mismatch can also occur. Furthermore, there is a case where the acceleration is not visually recognized although the vestibular organ senses the acceleration. In addition, there is a case where the vestibular organ does not sense the acceleration although the acceleration is visually recognized.

The mismatch between the vestibular information and the visual information may cause a motion sickness state. The mismatch of these pieces of information is notified to the amygdaloid complex of the limbic system, and it is determined whether the information is pleasant or unpleasant. In a case where these pieces of information are determined as unpleasant information, the motion sickness state may occur. For example, the unpleasant information stimulates the sympathetic nervous system via the hypothalamus, and secretes stress hormones into the body. The stress hormones may cause a reaction of a human body indicating the motion sickness state. The reactions of the human body include, for example, pallor of the face, cold sweat, and secretion of saliva. When the motion sickness state further proceeds, vomiting or a large fluctuation of the blood pressure may occur. In this way, in a case where a human will be in the motion sickness state or is in the motion sickness state, various biological reactions occur.

Furthermore, in addition to the mismatch of the pieces of information, for example, an environmental cause around the human such as an odor, temperature, or humidity may cause the motion sickness state. For example, smell in an automobile may cause a motion sickness.

(1-2) Details of First Embodiment

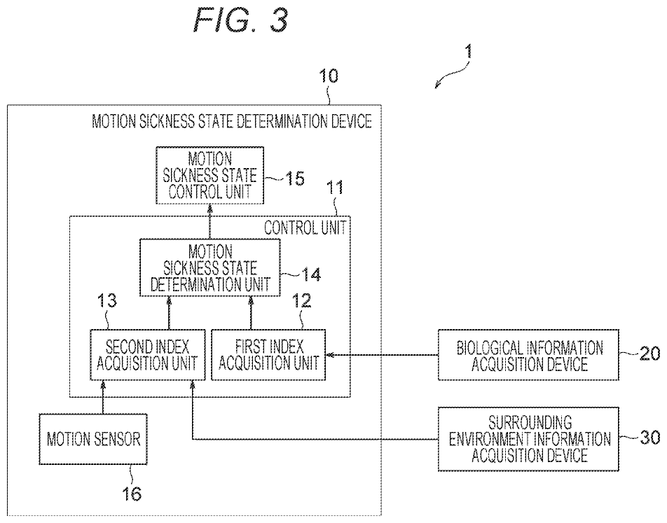

A schematic block diagram of an example of a motion sickness state determination system according to the first embodiment of the present technology is illustrated in FIG. 3. A motion sickness state determination system 1 illustrated in FIG. 3 includes a motion sickness state determination device 10, a biological information acquisition device 20, and a surrounding environment information acquisition device 30. Each of these devices will be described below.

The motion sickness state determination device 10 includes a control unit 11. The control unit 11 includes a first index acquisition unit 12, a second index acquisition unit 13, and a motion sickness state determination unit 14. The motion sickness state determination device 10 may further include or does not need to include a motion sickness state control unit 15. The motion sickness state determination device 10 may further include or does not need to include a motion sensor 16.

The control unit 11 may include, for example, a hard disk, a CPU or MPU, and a memory that store programs and an OS used to make the motion sickness state determination device 10 execute motion sickness state determination processing to be described below herein. For example, a function of the control unit 11 may be implemented by a general-purpose computer. The program may be recorded in a recording medium, for example, a microSD memory card, an SD memory card, a flash memory, or the like. A drive included in the motion sickness state determination device 10 may read the program recorded in the recording medium, and then, the control unit 11 may make the motion sickness state determination device 10 execute the motion sickness state determination processing according to the present technology in accordance with the read program.

The first index acquisition unit 12 acquires biological information as a first motion sickness state index. As described above, in a case where a human will be in the motion sickness state or is in the motion sickness state, various biological reactions occur. Information regarding these biological reactions may be acquired as the first motion sickness state index.

The biological information may be the biological information acquired by the biological information acquisition device 20 to be described later, and the motion sickness state determination device 10 may receive the biological information from the biological information acquisition device 20.

The second index acquisition unit 13 acquires a second motion sickness state index on the basis of movement information regarding a movement of a subject and/or surrounding environment information perceived by the subject.

The motion sensor 16 acquires the movement information. As the motion sensor 16, for example, an acceleration sensor and/or a gyro sensor may be used, and more preferably, an acceleration sensor is used. The acceleration sensor is preferably a three-axis acceleration sensor, and the acceleration sensor can detect an acceleration similar to an acceleration sensed by the vestibular organ. The motion sensor 16 may be configured as a part of the motion sickness state determination device 10 or the surrounding environment information acquisition device 30, and more preferably, may be configured as a part of the motion sickness state determination device 10. The motion sickness state determination device 10 may receive the movement information from the motion sensor.

FIG. 3 illustrates a mode in which the motion sickness state determination device 10 includes the motion sensor 16. The motion sensor 16 may be configured as a device different from the motion sickness state determination device 10.

Furthermore, the surrounding environment information is used to acquire the second motion sickness state index. However, in a case where the movement information is not used, the motion sickness state determination system 1 does not need to include the motion sensor 16. For example, in a case where the second motion sickness state index is acquired on the basis of only odor information, the motion sickness state determination system 1 does not need to include the motion sensor.

The surrounding environment information is information regarding an environment around the subject and may be, for example, information regarding surrounding information visually perceived and/or perceived by sense of smell by the subject.

The surrounding environment information that is visually perceived may be, for example, video information (also referred to as “video data”) visually recognized by the subject. The video information may be a video of an external landscape or may be a video presented to the subject from a display (particularly, head mounted display). The video of the external landscape may be acquired by, for example, an image sensor. The image sensor may be, for example, a CMOS or a CCD.

The surrounding environment information perceived by sense of smell may be, for example, an odor. The odor may be acquired by, for example, an odor sensor. The odor sensor can include, for example, a conductometric type odor sensor (chemoresistor), a capacitive type odor sensor (chemocapacitor), a potentiometric type odor sensor (chemodiode, chemotransistor, or the like), a calorimetric type odor sensor (thermo-chemosensor), a gravimetric type odor sensor, an optical type odor sensor, and an electrochemical type odor sensor, and any one of these odor sensors may be used.

In a case where the surrounding environment information is the video of the external landscape, the surrounding environment information acquisition device 30 including the image sensor acquires video information of the external landscape, and the surrounding environment information acquisition device 30 may transmit the video information of the external landscape to the motion sickness state determination device 10.

In a case where the surrounding environment information is the video presented to the subject from the display, the surrounding environment information acquisition device 30 including the image sensor may acquire the video information presented by the display, or the surrounding environment information acquisition device 30 may acquire the video information, for example, from the display or a video processing device that controls the display, or the like. The surrounding environment information acquisition device 30 may transmit the video information to the motion sickness state determination device 10.

In a case where the surrounding environment information is odor information, the surrounding environment information acquisition device 30 including the odor sensor may acquire the odor information. The surrounding environment information acquisition device 30 may transmit the odor information to the motion sickness state determination device 10.

As described above, the motion sickness state may be triggered by the mismatch between the vestibular information and the visual information. According to one embodiment of the present technology, the second index acquisition unit may acquire, for example, information regarding the mismatch on the basis of the movement information and the surrounding environment information as a second motion sickness state index. In this embodiment, in addition to the information regarding the mismatch, the odor information may be acquired as the second motion sickness state index.

Furthermore, the motion sickness state may be triggered by, for example, an odor. According to the other embodiment of the present technology, the second index acquisition unit may acquire, for example, the information regarding the odor as the second motion sickness state index on the basis of the surrounding environment information.

The motion sickness state determination unit 14 determines whether or not the subject will be in a motion sickness state or is in the motion sickness state on the basis of the first motion sickness state index and the second motion sickness state index. By determining the motion sickness state of the subject on the basis of these two indexes, an effect is obtained such that the motion sickness state of each subject can be more accurately determined. The effect will be specifically described below.

In the motion sickness state, as described above, biological reactions such as sweating, pallor of the face, or the like may occur. However, the biological reaction does not necessarily indicate a sign of a motion sickness. In the motion sickness state determination system according to the present technology, the first index acquisition unit acquires the biological information, and in addition, the second index acquisition unit acquires the second motion sickness state index on the basis of the movement information and/or the surrounding environment information. By determining the motion sickness state on the basis of the second motion sickness state index in addition to the first motion sickness state index, it is possible to more accurately determine a motion sickness state of each subject.

Furthermore, as described above, the motion sickness state may be triggered by the mismatch between the vestibular information and the visual information. However, even if the mismatch occurs, for example, there is a case where the subject is not in the motion sickness state depending on the constitution or physical condition of the subject. In the motion sickness state determination system according to the present technology, the second index acquisition unit detects, for example, the mismatch between the vestibular information and the visual information, and in addition, the first index acquisition unit acquires the biological information as the first motion sickness state index. By determining the motion sickness state on the basis of the first motion sickness state index in addition to the second motion sickness state index, it is possible to more accurately determine the motion sickness state of each subject.

As described above, in a case where both of the first motion sickness state index and the second motion sickness state index satisfy references set for the respective indexes, the motion sickness state determination unit 14 may determine that the subject will be in the motion sickness state or is in the motion sickness state.

As described above, the motion sickness state determination system according to the present technology can accurately determine the motion sickness state of each subject. Therefore, the determination result makes it possible to more appropriately cope with the motion sickness state according to the individual subject as described below.

As described above, the motion sickness state may be triggered by the mismatch between the vestibular information and the visual information. In the motion sickness state determination system according to the present technology, the second index acquisition unit may detect, for example, the mismatch between the vestibular information and the visual information. Therefore, it is possible to apply a stimulus to correct the mismatch to the subject.

Furthermore, as described above, even if the mismatch occurs, for example, there is a case where the subject is not in the motion sickness state depending on the constitution or the physical condition of the subject. In the motion sickness state determination system according to the present technology, the first index acquisition unit acquires the biological information as the first motion sickness state index. In a case where it is determined that the subject is not in the motion sickness state on the basis of the biological information, it is not necessary to apply the stimulus to correct the mismatch to subject, and it is possible to prevent the unnecessary stimulus to the subject in this case.

From the viewpoint of appropriate coping with the motion sickness state described above, the motion sickness state determination system according to the present technology may further include the motion sickness state control unit 15 that controls the motion sickness state of the subject on the basis of the determination result of the motion sickness state determination unit. Content of processing executed by the motion sickness state control unit is based on the determination result of the motion sickness state determination unit. Therefore, as described above, more accurate countermeasures can be taken according to each subject.

The motion sickness state control unit 15 may determine content of processing for controlling the motion sickness state, preferably, on the basis of information acquired from a database. The database may be included in the motion sickness state determination device 10 or may be outside the motion sickness state determination device 10. The database may record, for example, use information of the motion sickness state determination device 10 by the subject. The database may be used as a search database for searching the content of the processing executed by the motion sickness state control unit 15. The database may record use information of the motion sickness state determination device 10 by other subject. On the basis of these pieces of use information, it is possible to optimize the content of the processing executed by the motion sickness state control unit 15. Therefore, it is possible to more effectively control the motion sickness state. Moreover, it is possible to suppress or eliminate side effects caused by the motion sickness state control.

The use information includes, for example, the first motion sickness state index, the second motion sickness state index, the determination result by the motion sickness state determination unit, the content of the processing executed by the motion sickness state control unit 15 on the basis of the determination result, and the biological information of the subject after the processing. However, the use information is not limited to these.

The database may record physical information of various subjects other than the subject, in addition to physical information of a subject who uses this system. The physical information may include, for example, at least one or a combination of two or more pieces of information selected from a group including a gender, an age, a height, and a weight. For example, the motion sickness state control unit 15 may optimize the processing content on the basis of the use information of the other subject having the physical information similar to the subject who uses the motion sickness state determination device 10.

Particularly, the database is preferably a cloud database. The cloud database may accumulate the use information as needed. Then, more appropriate processing may be selected on the basis of the accumulated database.

As the processing for controlling the motion sickness state, for example, the motion sickness state control unit 15 may apply a stimulus to prevent or suppress the motion sickness state to the subject, apply a stimulus to trigger the motion sickness state to the subject, or improve surrounding environment that causes the motion sickness state. The processing executed by the motion sickness state control unit 15 may include, for example, one, two, or three selected from among application of a current to the subject, presentation of an odor to the subject, removal of the an odor around the subject. The application of the current may be preferably Galvanic Vestibular Stimulation (also referred to as GVS below). The GVS is to apply a weak external current of several mA to the vestibule, and this can give an illusion of the acceleration to the brain. A more specific example of the processing executed by the motion sickness state control unit 15 will be described in (2) to (7) below.

The motion sickness state determination device 10 preferably determines the motion sickness state of the subject in real time. For example, motion sickness state determination processing described below is continuously or periodically executed. With this processing, the motion sickness state can be accurately determined according to a change in environment where the subject who uses the motion sickness state determination device 10 is positioned or a change in the subject, and it is possible to execute more appropriate treatment to prevent or suppress the motion sickness state.

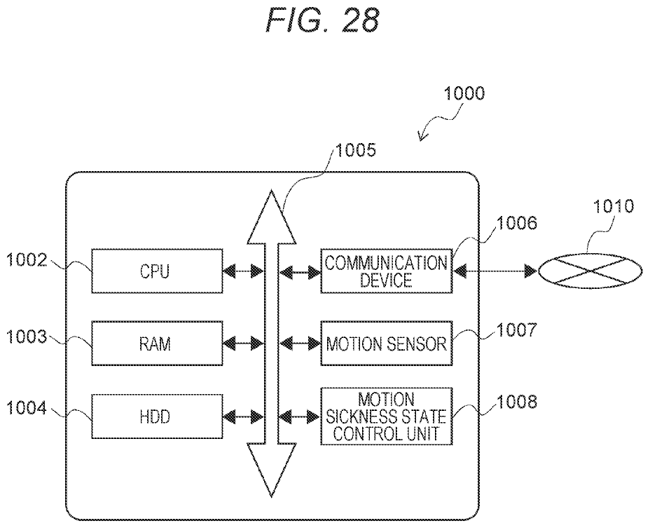

A hardware configuration example of the motion sickness state determination device according to the present technology will be described below with reference to FIG. 28.

A motion sickness state determination device 1000 illustrated in FIG. 28 includes a Central Processing Unit (CPU) 1002 and a RAM 1003. The CPU 1002 and the RAM 1003 are connected to each other via a bus 1005 and are connected to other components of the motion sickness state determination device 1000 via the bus 1005.

The CPU 1002 controls and operates the motion sickness state determination device 1000. Any processor may be used as the CPU 1002. The function of the control unit 11 of the motion sickness state determination device 10 described with reference to FIG. 3 may be implemented, for example, by the CPU 1002.

The RAM 1003 includes, for example, a cache memory and a main memory and may temporarily store a program used by the CPU 1002 or the like.

The motion sickness state determination device 1000 may include a disk 1004, a communication device 1006, a motion sensor 1007, and a motion sickness state control unit (for example, current application device) 1008. The motion sickness state determination device 1000 includes a drive as needed, and each of these components may be connected to the bus 1005.

The disk 1004 may store programs for implementing a motion sickness state determination method according to the present technology such as an operating system (for example, WINDOWS (registered trademark)), UNIX (registered trademark), or LINUX (registered trademark), various other programs, and various types of data.

The communication device 1006 wiredly or wirelessly connects the motion sickness state determination device 1000 to a network 1010. The communication device 1006 can acquire various types of databases (for example, database (in particular, cloud database), data transmitted from biological information acquisition device 20 and surrounding environment information device 30 or the like) via the network 1010. The acquired data may be stored, for example, in the disk 1004. The type of the communication device 1006 may be appropriately selected by those skilled in the art. The disk 1004 may be, for example, a semiconductor recording medium such as a flash memory, and is not limited to this.

The motion sensor 1007 may acquire the movement information used to acquire the second motion sickness state index.

The drive can read information recorded in a recording medium and output the read information to the RAM 1003. The recording medium is, for example, a microSD memory card, a SD memory card, or a flash memory. However, the recording medium is not limited to this.

Preferably, the biological information acquisition device 20 may acquire biological information that appears or fluctuates in a case where the subject will be in the motion sickness state or is in the motion sickness state. The biological information may be, for example, one or a combination of two or more pieces of information selected from a group including the information regarding the enzyme derived from the subject (for example, enzyme amount, enzyme activity, or the like), respiration information (for example, respiration rate, inhalation time, exhalation time, or the like), face color information (for example, degree of pallor of face), saliva amount information, sweating information (for example, cold sweat, sweating amount, sweating or not, or the like), blood pressure information (for example, maximum blood pressure, diastolic blood pressure, or the like), and heart rate information (for example, heart rate or the like).

According to the preferred embodiment of the present technology, the biological information acquired by the biological information acquisition device 20 includes information regarding the enzyme derived from the subject, and more preferably, includes information regarding an activity and/or an amount of the enzyme derived from the subject. More preferably, the enzyme is an amylase. For example, the biological information may be an amylase activity and/or an amylase amount in saliva of the subject.

The amylase activity and the amylase amount in the saliva are preferable to reduce a size of a device included in the motion sickness state determination system, and are further preferable for accurate motion sickness state determination. For example, to detect pallor of the face, it is necessary to provide a distance between a detection device and the face from which the face can be determined. Therefore, it is difficult to reduce the size of the device that detects the pallor of the face, and in addition, it is difficult to form the device as a wearable device. Furthermore, it is difficult to distinguish a cold sweat from an environmental sweat. Furthermore, to determine that the sweat is a cold sweat by analyzing a sweat component, a large amount of sweats are needed. Therefore, it is difficult to reduce a size of a device that detects the cold sweat, and in addition, it is difficult to form the device as a wearable device. On the other hand, regarding saliva (saliva), medical studies indicate that it is possible to determine pleasant or unpleasant according to activity fluctuation of the amylase in a small amount of saliva (for example, Nakano and Yamaguchi, Japanese journal of biofeedback research Vol. 38, No. 1, 2011). Furthermore, the present inventor or the like have proposed a real-time monitoring method in the oral cavity regarding the amylase activity in the saliva (International publication No. WO 2016/042908 A and International publication No. WO 2018/066227 A). A device used for the real-time monitoring method is compact and can accurately measure the amylase activity.

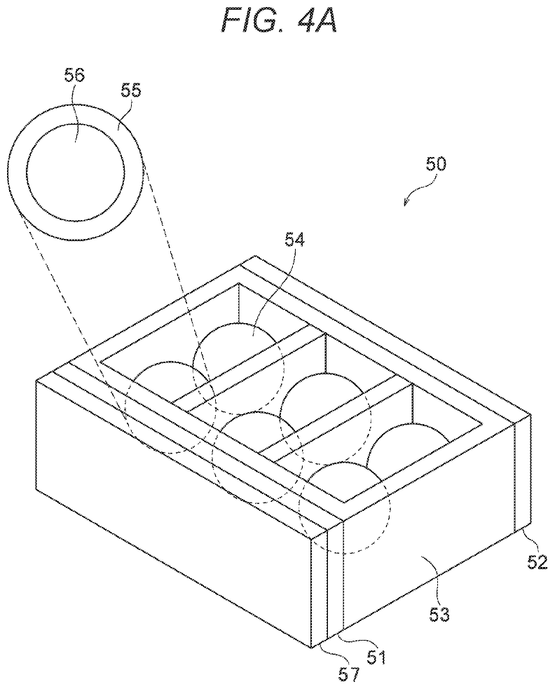

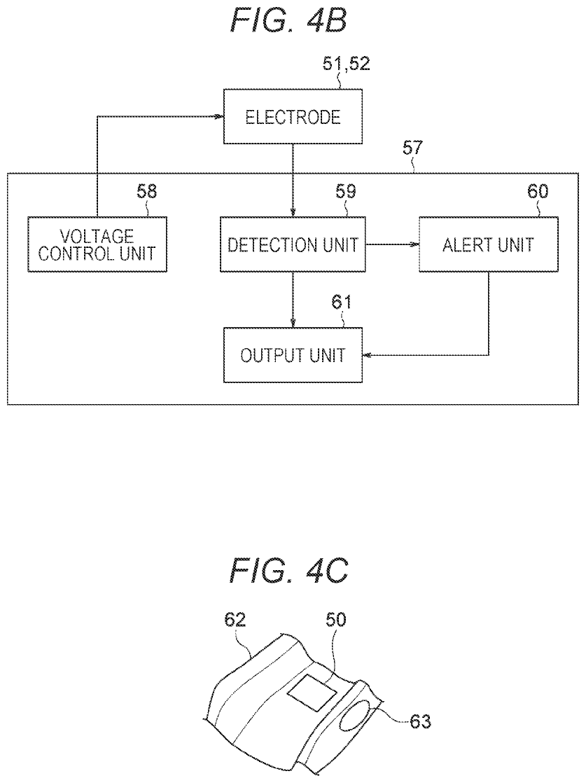

The biological information acquisition device 20 may preferably include an enzyme sensor and may more preferably include an amylase sensor. The information regarding the enzyme may be preferably acquired by an enzyme sensor, and more preferably, by a device for measuring an amount and/or an activity of the enzyme. As the enzyme sensor, for example, the enzyme sensor described in International publication No. WO 2018/066227 A mentioned above may be used. The enzyme sensor will be described below with reference to FIGS. 4A, 4B, and 4C. FIG. 4A is a schematic diagram of an example of the enzyme sensor. FIG. 4B is a block diagram of an example of the enzyme sensor. FIG. 4C is a diagram illustrating a configuration example of the enzyme sensor.

As illustrated in FIG. 4A, an enzyme sensor (device for measuring amount and/or activity of enzyme) 50 includes a pair of electrodes 51 and 52, an electron transfer layer 53 sandwiched by the pair of electrodes 51 and 52, and an electron generation capsule 54 that includes at least one or more types of enzymes other than the enzyme to be detected with a film that includes at least the substrate of the enzyme to be detected and has contact with the electron transfer layer 53. The electron generation capsule 54 may be held in the electron transfer layer 53 as illustrated in FIG. 4A. Moreover, the enzyme sensor 50 may include a logic unit 57.

The pair of electrodes 51 and 52 functions as an output unit that extracts electrons generated by a reaction with the enzyme to be detected as a detection signal. Specifically, the pair of electrodes 51 and 52 can extract the electrons generated by the electron generation capsule 54 via the electron transfer layer 53 by applying a voltage between the electrodes.

The pair of electrodes 51 and 52 may include a material having a conductivity. These electrodes may be provided as a pair of parallel plates sandwiching the electron transfer layer 53 therebetween. Specifically, the pair of electrodes 51 and 52 may include, for example, a metal or an alloy of copper (Cu), silver (Ag), platinum (Pt), aluminum (Al), tungsten (W), titanium (Ti), or the like or may include a carbon material, for example, graphite, amorphous carbon, or the like. More preferably, the pair of electrodes 51 and 52 may include a metal or an alloy that hardly causes corrosion such as rust or a carbon material so as to be suitable for use in an environment where the measurement device 50 has contact with fluid of a living body.

Regarding the one pair of electrodes 51 and 52, one electrode functions as an anode, and the other electrode functions as a cathode. However, the polarity of the pair of electrodes 51 and 52 may be appropriately set according to a detection signal extraction direction.

The electron transfer layer 53 has contact with the electron generation capsule 54 and is sandwiched by the pair of electrodes 51 and 52. The electron transfer layer 53 transfers electrons generated by the electron generation capsule 54 to one of the electrodes 51 and 52 (that is, anode). More specifically, the electron transfer layer 53 may include an electron transfer mediator. The electron transfer mediator may be, for example, an electron-acceptable electrolyte, and the electrolyte may transfer the electrons to the anode side that is one of the electrodes 51 and 52. The electron transfer layer 53 may be, for example, formed by gelling a solution including the electrolyte, and that is, the electron transfer layer 53 may be a gelled object of a solution that includes electrolytes.

The electrolyte included in the electron transfer layer 53 may be a known substance that can reversibly accept or emit electrons. As the electrolyte, for example, a substance including ferricyanide ions may be used, and for example, potassium ferricyanide can be used. The ferricyanide ion ([Fe (CN)₆]³⁻) can reversibly change to a ferrocyanide ion ([Fe (CN)₆]⁴⁻) through oxidation-reduction reaction. That is, the ferricyanide ion ([Fe (CN)₆]³⁻) changes to the ferrocyanide ion ([Fe (CN)₆]⁴⁻) to accept the electrons, and returns to the ferricyanide ion ([Fe (CN)₆]³⁻) with either one of the electrodes 51 and 52 so as to emit the electrons. Other electron transfer mediators include, for example, benzoquinone.

In the electron transfer layer 53, at least one or more recess structures are provided on a surface that is not sandwiched by the pair of electrodes 51 and 52, and the electron generation capsule 54 may be held in the recess structure. By providing the recess structure in the electron transfer layer 53, the electron transfer layer 53 can reliably hold the electron generation capsule 54. Furthermore, with the recess structure, it is possible to increase a contact area between the electron transfer layer 53 and the electron generation capsule 54.

The plurality of recess structures may be provided in the electron transfer layer 53, and the plurality of electron generation capsules 54 may be held in the recess structure. By increasing the number of recess structures, it is possible to increase the contact area between the electron transfer layer 53 and the electron generation capsule 54. Furthermore, by increasing the number of electron generation capsules 54 in the recess structure, it is possible to increase the number of reaction sites between the enzyme and the electron generation capsule 54. The number of recess structures provided in the electron transfer layer 53 and the number of electron generation capsules 54 provided in the recess structure may be appropriately set.

The electron generation capsule 54 has a reaction site that generates electrons through an enzyme reaction with the enzyme to be detected and generates electrons according to the amount of the enzymes to be detected. Specifically, the electron generation capsule 54 has a structure in which an outer membrane 55 that includes at least a substrate of the enzyme to be detected contains an inner solution 56 that includes one or more types of enzymes other than the enzyme to be detected.

The outer membrane 55 includes the substrate of the enzyme to be detected and functions as the reaction site of the enzyme sensor 50 with the enzyme to be detected. Therefore, a shape of the electron generation capsule 54 specified by the outer membrane 55 may be a substantially spherical shape so as to increase an area of a surface that is the reaction site with the enzyme to be detected.

The outer membrane 55 functions to retain the inner solution 56 in the electron generation capsule 54 so that the inner solution 56 does not leak to the outside of the enzyme sensor 50. Therefore, in order to strengthen the outer membrane 55 as a film, the outer membrane 55 includes a membrane constituent, for example, lipids, proteins, carbohydrates, or the like in addition to the substrate of the enzyme to be detected.

The enzyme included in the inner solution 56 may include at least an oxidation enzyme. The substrate of the oxidation enzyme is a product generated by enzyme reactions of the substrate included in the outer membrane 55 including at least one or more stages. This makes it possible to extract the electrons from the product by oxidizing the product generated by the reaction of the enzyme to be detected by the oxidation enzyme. Furthermore, the enzymes included in the inner solution 56 may include the enzyme to be detected and the enzyme using the product of the reaction with the substrate contained in the outer membrane 55 as a substrate. At this time, the oxidation enzyme included in the inner solution 56 uses a final product of the reaction, including a plurality of stages, of the enzyme to be detected and the enzyme included in the inner solution 56 as a substrate.

The inner solution 56 may further include a solvent in which the enzyme is dissolved (for example, water), a functional polymer that stabilizes the enzyme, or the like. For example, the inner solution 56 may further include water dispersions of PMEH (copolymer of 2-Methacryloyloxyethyl phosphorylcholine and 2-Ethylhexyl methacrylate) with high biological compatibility.

Specifically, the reaction between the electron generation capsule 54 and the enzyme to be detected proceeds as follows. First, the enzyme to be detected reacts with the substrate included in the outer membrane 55, and an enzyme reaction product of the substrate is generated. Subsequently, the generated enzyme reaction product is oxidized by the oxidation enzyme included in the inner solution 56 exuded from the inside due to a difference in an osmotic pressure and emits electrons. The emitted electron is accepted by the electrolyte included in the electron transfer layer 53 and extracted by one of the electrodes 51 and 52 as described above.

For example, in a case where the enzyme to be detected is an amylase, the outer membrane 55 of the electron generation capsule 54 may include at least amylum that is the substrate of the amylase, and the inner solution 56 may include at least maltase and glucose oxidase.

In such a case, first, the amylase to be detected reacts with the amylum included in the outer membrane 55, and a maltose is generated. Next, the maltose included in the inner solution 56 exuded to the outer membrane 55 due to the difference in the osmotic pressure reacts with the maltase so as to generate glucose. Subsequently, glucose oxidase included in the inner solution 56 reacts with the glucose, and electrons are extracted from the glucose. Furthermore, the electrons extracted from the glucose are accepted by the electron transfer mediators, for example, ferricyanide ions or the like included in the electron transfer layer 53. For example, the ferricyanide ion accepts the electrons to be a ferrocyanide ion.

Furthermore, the enzyme sensor 50 may further include an ion exchange membrane that covers at least an opening of the recess structure of the electron transfer layer 53. The ion exchange membrane prevents the electron generation capsule 54, the component of the electron generation capsule 54, or the component of the electron transfer layer 53 from being leaked from the enzyme sensor 50 into the fluid.

In a case where the electron transfer mediator included in the electron transfer layer 53 is an anion (for example, ferricyanide ion), the ion exchange membrane may be a cation ion exchange membrane in order to prevent leakage of the anion included in the electron transfer layer 53. Furthermore, in a case where the electrolyte included in the electron transfer layer 53 is a cation, the ion exchange membrane may be an anion ion exchange membrane.

The enzyme sensor 50 may further include the logic unit 57. The logic unit 57 includes, for example, a voltage control unit 58, a detection unit 59, an alert unit 60, and an output unit 61.

The logic unit 57 may be implemented, for example, by cooperation of hardware such as a central processing unit (CPU), a random access memory (RAM), a read only memory (ROM), or the like and software that controls an operation of each component.

The voltage control unit 58 controls a voltage applied between the pair of electrodes 51 and 52 of the enzyme sensor 50 to measure the amount of the enzyme to be detected. Specifically, the voltage control unit 58 controls a voltage so that a rectangular pulse voltage is applied between the pair of electrodes 51 and 52.

The detection unit 59 detects the amount of the enzyme to be detected by measuring a current flowing between the pair of electrodes 51 and 52 of the enzyme sensor 50. Specifically, in a case where the rectangular pulse voltage is applied between the electrodes 51 and 52 by the voltage control unit 58, the electrolyte that has accepted the electrons of the electron transfer layer 53 (for example, ferrocyanide ion) emit the electrons to the anode-side electrode and returns to an electrolyte that does not accept the electrons (for example, ferricyanide ion). At this time, the emitted electrons cause the current to flow between the pair of electrodes 51 and 52.

Therefore, the detection unit 59 can detect an amount of the electrolyte that has accepted the electrons (for example, ferrocyanide ion) by detecting a magnitude of the current flowing between the pair of electrodes 51 and 52. Moreover, the amount of the electrolyte that has accepted the electrons (for example, ferrocyanide ion) depends on an amount of reaction between the enzyme to be detected and the electron generation capsule 54. Therefore, the detection unit 59 can calculate an amount of the enzyme to be detected from the magnitude of the current flowing between the pair of electrodes 51 and 52.

The alert unit 60 monitors a consumption of the substrates included in the outer membrane 55 of the electron generation capsule 54 and determines a timing of exchange of the enzyme sensor 50. Because the substrate included in the outer membrane 55 of the electron generation capsule 54 is consumed by the reaction with the enzyme to be detected, the substrate is reduced as the measurement by the enzyme sensor 50 proceeds. Therefore, in order to continue the measurement by the enzyme sensor 50, it is required to exchange the enzyme sensor 50 before all the substrates included in the outer membrane 55 of the electron generation capsule 54 are consumed. Specifically, the alert unit 60 determines the timing of the exchange of the enzyme sensor 50 on the basis of an integrated value of the measured current detected by the detection unit 59. Furthermore, the alert unit 60 may notify the subject to exchange the enzyme sensor 50 by display, voice, or a signal via the output unit 61.

The output unit 61 outputs information based on a current value detected by the detection unit 59 to the subject or an external device. For example, the output unit 61 may output information regarding an enzyme activity on the basis of the current value detected by the detection unit 59. Specifically, the output unit 61 may measure a current value corresponding to an amount of the enzyme at a normal time as a “reference current value” and perform calculation on the basis of the following formula so as to calculate the “enzyme activity” from the detected “measured current value” and output the calculated “enzyme activity”.

Enzyme activity=(measured current value−reference current value)/reference current value

The amount of the enzyme to be secreted individually varies for each living body. Therefore, by indicating the enzyme activity with an increase/decrease of a ratio with respect to the amount of the enzyme at the normal time as described above, it is possible to more accurately indicate the degree of the enzyme secretion amount of the living body to be a subject.

The output unit 61 may output the current value detected by the detection unit 59, not the activity of the enzyme to be detected. In such a case, another device (for example, motion sickness state determination device to be described later) that has acquired the output current value may calculate the activity of the enzyme to be detected.

For example the output unit 61 may output information to an external display device or a sound output device, for example, via a wired or wireless local area network (LAN), the Bluetooth (registered trademark), the Wi-Fi (registered trademark), or the like.

The enzyme sensor 50 may be mounted in a mouthpiece 62, for example, as illustrated in FIG. 4C. Furthermore, in addition, various modules such as an acceleration sensor, a global navigation satellite system (GNSS) sensor, a radio frequency (RF) module, a power generation module, and a power storage module may be mounted in the mouthpiece 62. Note that an orifice 63 may be formed in the mouthpiece 62 so that the enzyme sensor 50 can have contact with saliva.

According to a preferred embodiment of the present technology, the movement information is movement information regarding a movement of a body of the subject, particularly, a movement of the head, the surrounding environment information is video information regarding a video that is visually perceived by the subject, and the second index acquisition unit may acquire a second motion sickness state index on the basis of the movement information and the video information. In this embodiment, preferably, the movement information is first acceleration information based on the movement of the body of the subject, the surrounding environment information is second acceleration information based on the video visually perceived by the subject, and the second index acquisition unit may acquire a second motion sickness state index on the basis of the first acceleration information and the second acceleration information. More preferably, the second index acquisition unit may acquire a difference between the first acceleration information and the second acceleration information as the second motion sickness state index.

This embodiment will be described in more detail with reference to a system configuration example and a flow of determination processing in (2) to (6) below.

According to another preferred embodiment of the present technology, the surrounding environment information is odor information regarding an odor perceived by sense of smell by the subject, and the second index acquisition unit may acquire a second motion sickness state index on the basis of the odor information. In this embodiment, the second index acquisition unit may acquire an intensity of a predetermined type of odor as the second motion sickness state index. In this embodiment, this system may further include a motion sickness state control unit that presents an odor to the subject or removes an odor around the subject on the basis of a determination result of the motion sickness state determination unit.

This embodiment will be described in more detail with reference to a system configuration example and a flow of determination processing in (7) below.

More preferably, the motion sickness state determination system according to the present technology may further include an output unit that outputs the determination result of the motion sickness state determination unit. The output unit may output the determination result with an information transmission medium, for example, an image, sound, or a printed matter. By outputting the determination result via these media, it is possible to notify the subject of that the subject will be in a motion sickness state or in a motion sickness state.

(2) First Example of First Embodiment (Example of Determination Based on Amylase Activity and Acceleration)

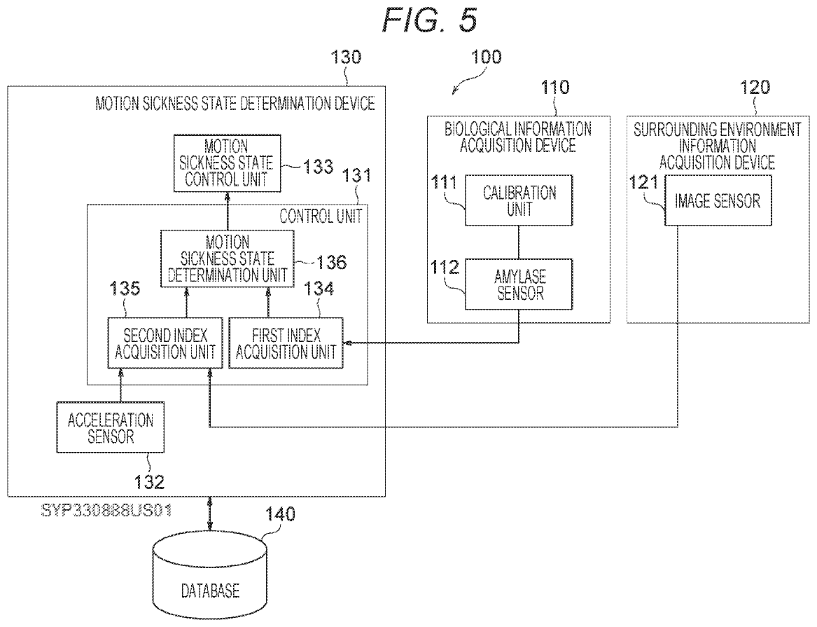

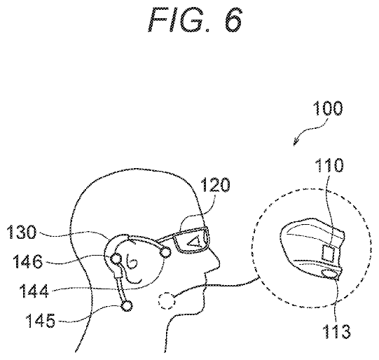

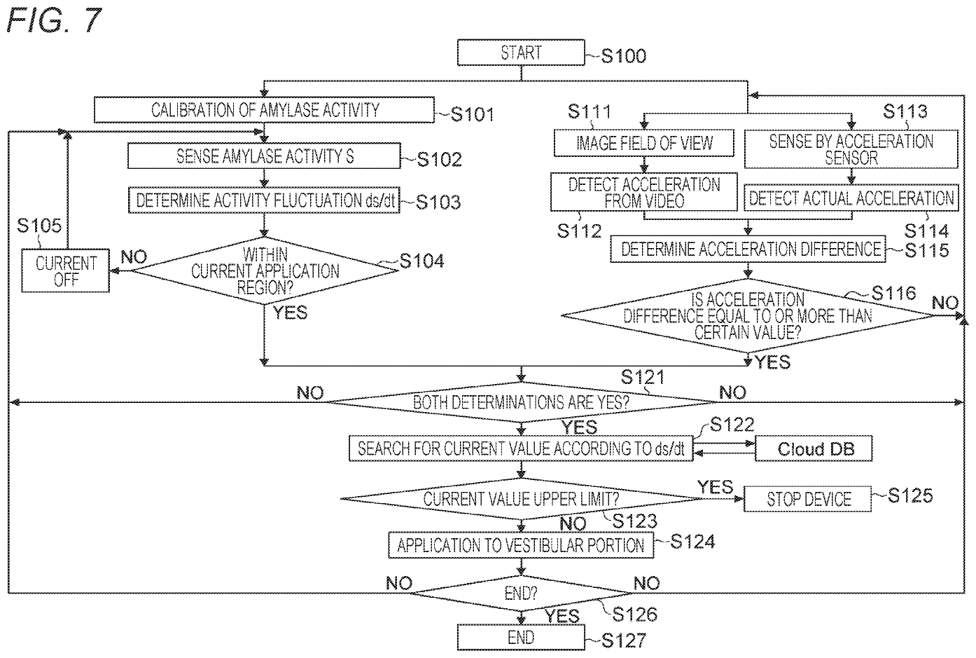

A block diagram of a motion sickness state determination system in this example is illustrated in FIG. 5, and a configuration example of a motion sickness state determination system in this example is illustrated in FIG. 6. Furthermore, an example of a flow of motion sickness state determination processing by the system is illustrated in FIG. 7. In this example, a first index acquisition unit acquires an amylase activity in saliva as a first motion sickness state index, and a second index acquisition unit acquires a difference in an acceleration acquired on the basis of movement information regarding a movement of the head of the subject and surrounding video information visually perceived by the subject as a second motion sickness state index.

In this example, as described above, it is determined whether or not the subject will be in the motion sickness state or is in the motion sickness state according to the saliva amylase activity and the acceleration difference. This makes it possible to more accurately determine the motion sickness state.

Furthermore, susceptibility to a motion sickness varies for each subject, and the susceptibility to a motion sickness of the same subject may vary depending on the physical condition of the subject. Therefore, by determining a motion sickness state by the motion sickness state determination system according to the present technology in real time, it is possible to more accurately determine and control the motion sickness state.

(2-1) Description of Configuration of Motion Sickness State Determination System of First Example

As illustrated in FIG. 5, a motion sickness state determination system 100 according to the present technology includes a biological information acquisition device (amylase detection device) 110 that measures an amylase activity in saliva, a surrounding environment information acquisition device (video acquisition device) 120 that acquires a video visually recognized by a subject who uses the system, and a motion sickness state determination device 130.

As illustrated in FIG. 6, the biological information acquisition device 110 is attached in an oral cavity of the subject so as to be able to have contact with saliva in the oral cavity of the subject. The surrounding environment information acquisition device 120 is attached near the eyes of the subject so as to acquire a video viewed by the subject. As illustrated in FIG. 6, the surrounding environment information acquisition device 120 may have, for example, a shape of an eyewear. The motion sickness state determination device 130 may be attached to the head of the subject, and preferably, attached near the ears of the subject so as to move in a same manner as the head of the subject. By being attached near the ears, an acceleration sensor 132 in the motion sickness state determination device 130 can detect a movement more closer to a movement sensed by the vestibular organ of the subject.

The biological information acquisition device 110 includes a calibration unit 111 and an amylase sensor 112. The biological information acquisition device 110 is configured to further include a communication unit (not illustrated) and to be wiredly or wirelessly communicable with the motion sickness state determination device 130 using the communication unit.

The biological information acquisition device 110 is configured as a partial mouthpiece, and more specifically, is configured as a mouthpiece worn in a back teeth portion. The amylase sensor 112 is disposed on an inner side (surface facing teeth or gum at the time of being worn) of the mouthpiece. An orifice 113 is provided in the mouthpiece, and the orifice 113 enables the amylase sensor 112 to have contact with saliva.

The calibration unit 111 performs calibration to measure the amylase activity by the amylase sensor 112. For example, immediately after or before start of the determination by the motion sickness state determination system 100, the calibration unit 111 may cause the amylase sensor 112 to execute calibration processing for acquiring an amylase activity to be a reference.

The amylase sensor 112 is the enzyme sensor described in “(1-2) details of first embodiment” described above. Because the description applies to the amylase sensor 112, the description regarding the amylase sensor 112 is omitted. The amylase sensor 112 may preferably perform monitoring in real time.

The surrounding environment information acquisition device 120 includes an image sensor 121. The surrounding environment information acquisition device 120 is configured to further include a communication unit (not illustrated) and to be wiredly or wirelessly communicable with the motion sickness state determination device 130 using the communication unit.

The surrounding environment information acquisition device 120 may be configured as an eyewear. The image sensor 121 is configured so as to acquire a video similar to an external landscape that is visually perceived by the subject who wears the eyewear. The image sensor 121 may be, for example, a CMOS or a CCD or may be an infrared (IR) sensor.

The motion sickness state determination device 130 includes a control unit 131, an acceleration sensor 132, and a motion sickness state control unit 133. The control unit 131 includes a first index acquisition unit 134, a second index acquisition unit 135, and a motion sickness state determination unit 136.

Furthermore, the motion sickness state determination device 130 may include a communication unit (not illustrated). The communication unit is configured to be communicable with the biological information acquisition device 110 and the surrounding environment information acquisition device 120 wirelessly or wirelessly, more preferably, wirelessly (for example, Bluetooth (registered trademark)).

The first index acquisition unit 134 acquires the amylase activity acquired by the amylase sensor 112 as a first motion sickness state index.

The acceleration sensor 132 may be, for example, a three-axis acceleration sensor. The acceleration sensor 132 detects a movement of a body of the subject, in particular, a movement of the head.

The second index acquisition unit 135 receives video information acquired by the surrounding environment information acquisition device 120. The second index acquisition unit 135 acquires an acceleration from the video information. The acceleration may be, for example, acceleration in a three-axis direction. The direction of the acquired acceleration may preferably correspond to a direction of the acceleration detected by the acceleration sensor 132. As a result, a more appropriate acceleration difference can be acquired as the second motion sickness state index.

The second index acquisition unit 135 acquires an acceleration difference between both accelerations as the second motion sickness state index on the basis of a first acceleration acquired from the video information acquired by the surrounding environment information acquisition device 120 and a second acceleration acquired by the acceleration sensor 132.

The motion sickness state determination unit 136 determines whether or not the subject will be in the motion sickness state or is in the motion sickness state on the basis of the first motion sickness state index and the second motion sickness state index. For example, the motion sickness state determination unit 136 may have a first threshold and a second threshold respectively to be compared with the first motion sickness state index and the second motion sickness state index in advance. In a case where the first motion sickness state index is equal to or more than the first threshold and the second motion sickness state index is equal to or more than the second threshold, the motion sickness state determination unit 136 may determine that the subject will be in the motion sickness state or is in the motion sickness state. Furthermore, in a case where the first motion sickness state index is not equal to or more than the first threshold, in a case where the second motion sickness state index is not equal to or more than the second threshold, or in a case where the first motion sickness state index and the second motion sickness state index are respectively not equal to or more than the first threshold and not equal to or more than the second threshold, the motion sickness state determination unit 136 may determine that the subject will not be in the motion sickness state or is not in the motion sickness state.

The motion sickness state control unit 133 is configured to be able to apply a current to the head (particularly, vestibular organ of inner ear) of the subject. For example, as illustrated in FIG. 6, the motion sickness state control unit 133 is disposed neat the right ear, and a motion sickness state control unit (not illustrated) is also disposed near the left ear. The motion sickness state control unit 133 disposed near the right ear includes three electrodes 144, 145, and 146 as illustrated in FIG. 6. These electrodes may be disposed, for example, at three positions including a position near the temple, a position in a neck portion under the back of the ear (or mastoid) (for example, two cm to 10 cm below, particularly, four cm to eight cm below, more particularly, about six cm below), and a position near the mastoid. The motion sickness state control unit disposed near the left ear may be similarly disposed at three positions. The GVS using the electrodes disposed in such a way can give the brain the illusion of the acceleration, and this makes it possible to control the motion sickness state.

The motion sickness state determination device 130 may be communicably connected to a database 140 wiredly or wirelessly. The database 140 may be, for example, a cloud database. The database 140 may store various types of data to be described in (2-2) below. The database 140 may be updated as needed.

(2-2) Description of Example of Determination Processing by Motion Sickness State Determination System of First Example

In step S100 in FIG. 7, the motion sickness state determination system 100 starts the motion sickness state determination processing. In step S100, the motion sickness state determination device 130 activates in response to a start signal of the processing input by the subject, and then, the motion sickness state determination device 130 transmits signals to activate the biological information acquisition device 110 and the surrounding environment information acquisition device 120 respectively to these devices. These devices activate in response to the receipt of the signal.

In step S101, the biological information acquisition device 110 (particularly, calibration unit 111) may cause the amylase sensor 112 to execute the calibration processing to acquire the amylase activity to be a reference. In the calibration processing, the amylase sensor 112 automatically measures the amylase activity, for example, for several minutes, and a reference value may be set on the basis of the measurement result.

In step S102, the biological information acquisition device 110 (particularly, amylase sensor 112) acquires amylase activity information. For example, the biological information acquisition device 110 measures an amylase activity S in saliva of the subject as the amylase activity information. The measurement may be preferably performed in real time. The biological information acquisition device 110 transmits the acquired amylase activity information to the motion sickness state determination device 130 through the communication unit.

In step S103, the motion sickness state determination device 130 receives the amylase activity information from the biological information acquisition device 110. The first index acquisition unit 134 of the motion sickness state determination device 130 calculates, for example, a variability rate dS/dt of the amylase activity S with respect to time from the received amylase activity information. The variability rate may be used by the motion sickness state determination unit 136 as the first motion sickness state index. Note that the amylase activity S itself may be used by the motion sickness state determination unit 136 as the first motion sickness state index. That is, in step S103, the first index acquisition unit 134 may acquire the variability rate or the amylase activity as the first motion sickness state index.

In step S104, the control unit 131 (for example, motion sickness state determination unit 136) determines whether or not the variability rate or the amylase activity is equal to or more than a threshold. Hereinafter, the threshold used for the determination in step S104 is referred to as a first threshold. The first threshold may be preset or may be updated as needed. The first threshold may be set or updated on the basis of data, for example, stored in the database 140, more specifically, in the cloud database. As the data used for the setting or update, for example, one or more pieces of information selected from among amylase activity information of the subject measured in the past, motion sickness state determination results of the subject in the past, physical information of the subject may be used. Furthermore, as the data, amylase activity information of humans other than the subject, motion sickness state determination results of humans other than the subject in the past, and physical information of humans other than the subject may be used.

In step S104, in a case where the variability rate or the amylase activity is equal to or more than the first threshold, the control unit 131 proceeds the processing to step S105. In a case where the variability rate or the amylase activity is less than the threshold, the control unit 131 proceeds the processing to step S121.

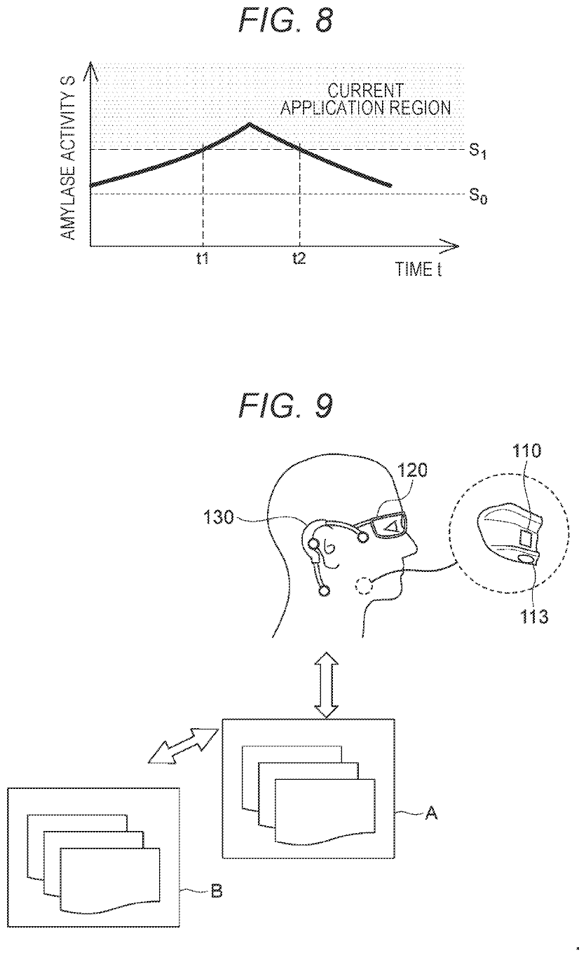

An example of the determination in step S104 will be described with reference to FIG. 8. FIG. 8 is a graph illustrating an example of a fluctuation of the amylase activity S. As time t elapses, the amylase activity S gradually increases from a reference value So set in step S101. Then, at a time t1, the amylase activity S reaches a first threshold S₁. A region where the amylase activity is equal to or more than S₁ is referred to as a current applicable region, and in a case where the amylase activity is in this region, it is determined that the current can be applied to the subject. Because the amylase activity S is less than the first threshold S₁ before the time t1, in step S104, the motion sickness state determination unit 136 proceeds the processing to step S121.

From the time t1 to a time t2, the amylase activity S is equal to or more than the first threshold S₁. Therefore, in step S104, the motion sickness state determination unit 136 proceeds the processing to step S105. After the time t1, for example, by applying the current as described below, the amylase activity S decreases. Then, after the time t2, the amylase activity S falls below the first threshold S₁.

Because the amylase activity S is less than the first threshold S₁ after the time t2, the motion sickness state determination unit 136 proceeds the processing to step S121 in step S104.

In step S105, the processing returns to step S102, and the processing in steps S102 to S104 may be executed again. Steps S102 to S105 are preferably repeated, and more preferably, may be performed in real time.

Furthermore, in a case where steps S102 to S104 are performed in a state where the motion sickness state control unit 133 applies the current to the subject, the motion sickness state control unit 133 stops the application of the current in step S105.

In step S111, the surrounding environment information acquisition device 120 acquires a video of an external landscape. The video includes at least a part of the external landscape viewed by the subject and may include, for example, at least an external landscape in the line-of-sight direction of the subject. More specifically, the image sensor 121 may acquire the video. The surrounding environment information acquisition device 120 transmits the acquired video information to the motion sickness state determination device 130 through the communication unit.

In step S112, the motion sickness state determination device 130 receives the video information from the surrounding environment information acquisition device 120. The second index acquisition unit 135 of the motion sickness state determination device 130 detects or calculates an acceleration on the basis of the received video information. In the example below, the acceleration acquired on the basis of the video in step S112 is referred to as a “first acceleration”. The first acceleration corresponds to the acceleration on the basis of the video viewed by the subject. The first acceleration may be an acceleration in one or two directions selected from among, for example, an acceleration in the front-back direction of the subject, an acceleration in the horizontal direction, and an acceleration in the vertical direction or may be all accelerations in these three directions.

In step S113, the acceleration sensor 132 included in the motion sickness state determination device 130 detects an acceleration. Hereinafter, the acceleration detected by the acceleration sensor in step S113 is referred to as a “second acceleration”. The second acceleration corresponds to the acceleration sensed by the vestibular organ of the subject. The acceleration sensor 132 transmits the detected second acceleration to the second index acquisition unit 135.

In step S114, the second index acquisition unit 135 acquires the second acceleration detected by the acceleration sensor 132. The second acceleration may be one or two accelerations selected from among, for example, an acceleration in the front-back direction of the subject, an acceleration in the horizontal direction, and an acceleration in the vertical direction or may be all accelerations in these three directions.

Preferably, the direction of the second acceleration may correspond to the direction of the first acceleration acquired in step S112.

In step S115, the second index acquisition unit 135 obtains a difference between the first acceleration acquired in step S112 and the second acceleration acquired in step S113. Preferably, a difference between the accelerations in the same direction is obtained. The second index acquisition unit 135 acquires the difference as the second motion sickness state index.

In step S116, the motion sickness state determination unit 136 determines whether or not the difference obtained in step S115 is equal to or more than a threshold. Hereinafter, the threshold used for the determination in step S116 is referred to as a second threshold. The second threshold may be preset or may be updated as needed. The second threshold may be set or updated on the basis of data, for example, stored in the database 140, more specifically, in the cloud database. As the data used for the setting or the update, for example, one or more pieces of information selected from among the difference between the first acceleration and the second acceleration acquired in the past, the motion sickness state determination result of the subject in the past, and the physical information of the subject may be used. Furthermore, as the data, a difference between a first acceleration and a second acceleration acquired in the past regarding humans other than the subject, a motion sickness state determination result of humans other than the subject in the past, physical information of humans other than the subject may be used.

In a case where the difference between the first acceleration and the second acceleration is equal to or more than the second threshold in step S116, the motion sickness state determination unit 136 proceeds the processing to step S121. In a case where the difference is less than the second threshold, the processing returns to steps S111 and S113, and the processing in steps S111 and S113 may be executed again.

In step S121, the motion sickness state determination unit 136 determines whether or not the variability rate or the amylase activity is equal to or more than the first threshold and the difference between the first acceleration and the second acceleration is equal to or more than the second threshold.

In a case where the variability rate or the amylase activity is equal to or more than the first threshold and the difference between the first acceleration and the second acceleration is equal to or more than the second threshold, the motion sickness state determination unit 136 determines that the subject will be in the motion sickness state or is in the motion sickness state and proceeds the processing to step S122.

In a case where the condition is not satisfied such that the amylase activity or the variability rate is equal to or more than the first threshold and the difference between the first acceleration and the second acceleration is equal to or more than the second threshold, the motion sickness state determination unit 136 determines that the subject will not be in the motion sickness state or is not in the motion sickness state and returns the processing to steps S102, S111, and S113.

In step S122, for example, the control unit 131 may select an electrode to which the current is applied and/or may select a magnitude of the current.

The electrode to which the current is applied may be selected, for example, on the basis of the difference between the first acceleration and the second acceleration. An electrode corresponding to a direction of the difference between the first acceleration and the second acceleration (for example, vertical direction, horizontal direction, or front-back direction of subject) may be selected. For example, there is an acceleration difference in the vertical direction of the subject, the electrode near the mastoid or the electrode in the neck portion is selected as the electrode to which the current is applied. In a case where there is an acceleration difference in the horizontal direction of the subject, the electrodes near the left and right temples are selected as the electrodes to which the current is applied. In a case where there is an acceleration difference in the front-back direction of the subject, the electrode near the mastoid and the electrode near the temple are selected as the electrodes to which the current is applied. Preferably, an electrode corresponding to a direction in which the difference is the largest is selected.

The magnitude of the current to be applied may be selected, for example, on the basis of the variability rate dS/dt of the amylase activity S or the amylase activity S. For example, the magnitude of the current to be applied may be dS/dt×C. C is a correction item and, for example, may be a preset value or may be derived on the basis of data in the database 140 (particularly, cloud database). For example, C may be corrected to be larger in a case where the degree of the decrease in the amylase activity S due to the applied current is small or may be corrected to be smaller in a case where the degree of the decrease is large.

In the present technology, the magnitude of the current to be applied may be, for example, 0.01 mA to three mA, more preferably, 0.1 mA to two mM, even more preferably, 0.5 mA to 1.5 mA. The current in this numerical range is suitable for suppressing or preventing the motion sickness state.

The current to be applied is preferably equal to or less than five Hz, more preferably, equal to or less than three Hz, and even more preferably, equal to or less than two Hz. As a result, a gentle current is applied to the subject, and it is possible to prevent the subject from feeling a tingling pain caused by the current.

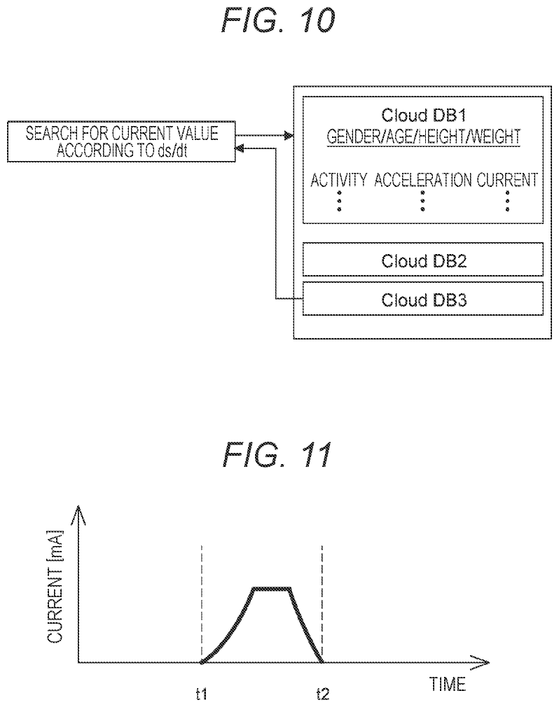

In step S122, as illustrated in FIG. 9, from a database A (particularly, cloud database) including data regarding the subject and/or a database B (particularly, cloud database) including data regarding humans other than the subject, the electrode to which the current is applied and/or the magnitude of the current to be applied may be searched. The database A may include one or more pieces of data selected from among, for example, physical information of the subject (for example, gender, age, height, weight, or the like), a motion sickness state determination result of the subject in the past, a current application history, a relationship between the acceleration difference and the amylase activity fluctuation, and the applied current and a response of the amylase activity caused by the current. As illustrated in FIG. 10, the database B may include one or more pieces of data selected from among, physical information of each human other than the subject (for example, gender, age, height, weight, or the like), the motion sickness state determination result of the humans other than the subject in the past, a current application history, a relationship between the acceleration difference and the amylase activity fluctuation, and the applied current and a response of the amylase activity caused by the current.

For example, in a case where the subject uses the motion sickness state determination system in this example for the first time, motion sickness state determination result data regarding the subject is not accumulated. Therefore, data regarding others such as the database B may be used. For example, from data regarding another person who has physical information closest to the physical information of the subject or data regarding a plurality of other persons having physical information close to the physical information of the subject, the electrode to which the current is applied and/or the magnitude of the current to be applied may be selected.

Such a database may be stored in the database 140.

In step S123, the control unit 131 determines whether or not the magnitude of the current selected in step S122 exceeds an upper limit value. The upper limit value may be preset or may be appropriately changed according to the subject. The upper limit value may be, for example, three mA, four mA, or five mA.

In a case where the magnitude of the current selected in step S122 does not exceed the upper limit value, the control unit 131 proceeds the processing to step S124.

In a case where the magnitude of the current selected in step S122 exceeds the upper limit value, the control unit 131 proceeds the processing to step S125.

In step S125, the control unit 131 may issue an error and stop the motion sickness state determination device 130. The error may be notified to the subject, for example, with sound or a video. Furthermore, along with the stop of the motion sickness state determination device 130, the biological information acquisition device 110 and/or the surrounding environment information acquisition device 120 may be stopped.

In step S124, the control unit 131 may drive the motion sickness state control unit 133 and apply a current stimulus to the subject. In this example, the motion sickness state control unit 133 may apply, for example, a current having the magnitude selected in step S123 to the electrode selected in step S123. With this process, the current may be applied to the vestibular organ of the subject. Therefore, the difference between the first acceleration and the second acceleration can be reduced or eliminated, and it is possible to prevent to be in the motion sickness state or to suppress the motion sickness state. The application of the current may be stopped, for example, after a predetermined time has elapsed, or may be stopped in response to that dS/dt or S falls below the first threshold. After the stop of the current application, the processing in steps S102, S111, and S113 may be executed again.

Alternatively, while the current is continuously applied, the processing in steps S102, S111, and S113 may be executed again.

After the application of the current in step S124, the control unit 131 proceeds the processing to step S126.

Specific examples of the current to be applied in step S124 will be described with reference to FIGS. 8 and 11. FIG. 8 is as described above. FIG. 11 is a diagram illustrating an example of a change in a current to be applied. In FIG. 8, at and after the time t1, the amylase activity S is equal to or more than the first threshold S1. Therefore, a current may be applied. As illustrated in FIG. 11, a current value may gradually increase from zero mA to a current value A1 that is the selected magnitude described above. As a result, it is possible to prevent the subject from feeling a pain caused by the current. Furthermore, the current value may be set so as not to exceed the upper limit value mentioned for step S123. Therefore, for example, as illustrated in FIG. 11, when the current value reaches the upper limit value, the increase in the current value stops, and then, the current value may be maintained at the upper limit value. Thereafter, the current value decreases, and for example, the current value may be zero in response to that the amylase activity S falls below the first threshold S1 at the time t2 in FIG. 8.

In step S126, the control unit 131 determines whether or not to end the motion sickness state determination processing. For example, in a case where a signal according to an operation for ending the processing is detected, it is determined to end the processing. In a case where it is determined to end the processing, the control unit ends the processing. In a case where the signal is not detected, the control unit 131 returns the processing to steps S102, S111, and S113, and the motion sickness state determination processing may be repeated again. In this way, in the present technology, the motion sickness state determination processing is repeated, and more preferably, executed in real time. The susceptibility to a motion sickness varies for each subject, and in addition, the susceptibility to a motion sickness of the same subject may vary depending on the physical condition of the subject. By repeating the motion sickness state determination processing, more preferably, by executing the processing in real time, it is possible to more accurately determine and control the motion sickness state.

According to the above processing, for example, in a case where the subject is in a vehicle or an aircraft, it is possible to appropriately determine the motion sickness state. Because the motion sickness state determination system in this example can appropriately determine the motion sickness state as described above, for example, it is also possible to notify the subject of that the subject will be in the motion sickness state or is in the motion sickness state. For example, in a case where an occupant of these vehicles is reading or watching TV, the acceleration difference described above may cause the motion sickness state. The above notification can urge the occupant to stop reading or watching TV. Furthermore, because the motion sickness state determination system in this example can apply an appropriate stimulus (current) according to the acceleration difference to the subject, it is also possible to prevent to be in the motion sickness state or to suppress the motion sickness state.

(3) Second Example of First Embodiment (Application Example to VR Devices)

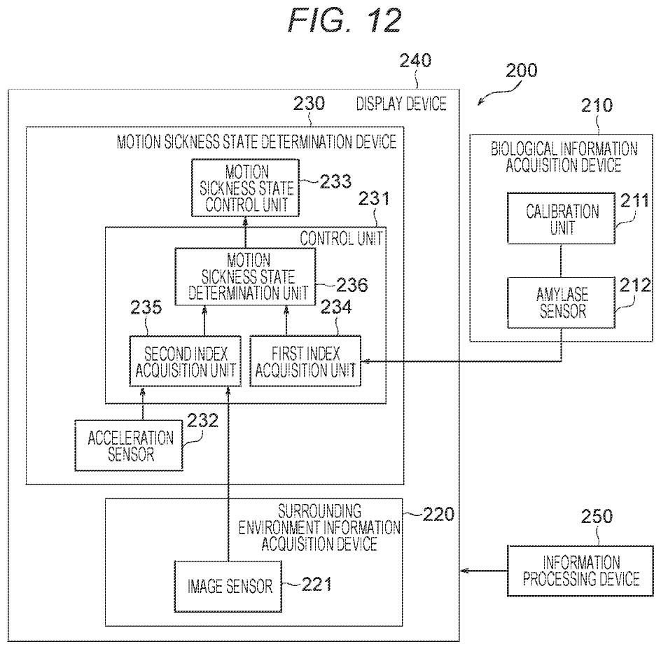

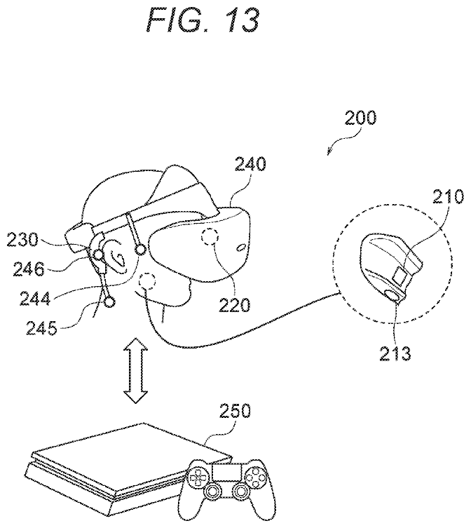

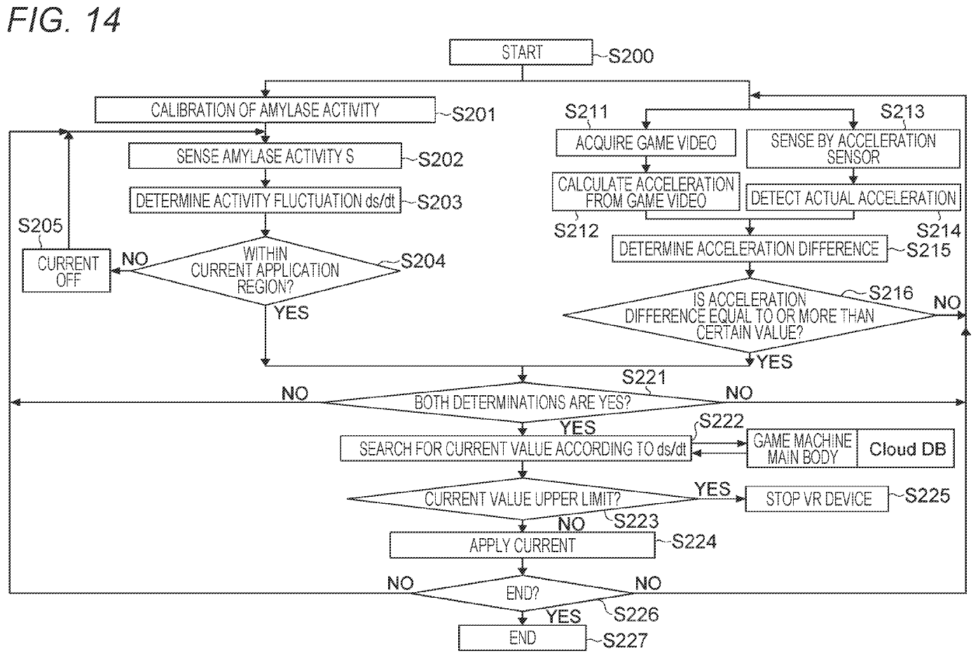

A block diagram of a motion sickness state determination system in this example is illustrated in FIG. 12, and a configuration example of the system is illustrated in FIG. 13. Furthermore, an example of a flow of motion sickness state determination processing by the system is illustrated in FIG. 14. In this example, a first index acquisition unit acquires an amylase activity in saliva as a first motion sickness state index, and a second index acquisition unit acquires a difference between accelerations acquired on the basis of movement information regarding a movement of the head of the subject and surrounding video information visually perceived by the subject as a second motion sickness state index.

(3-1) Description of Configuration of Motion Sickness State Determination System of Second Example

As illustrated in FIG. 12, a motion sickness state determination system 200 according to the present technology includes a biological information acquisition device (amylase detection device) 210 that measures an amylase activity in saliva, a surrounding environment information acquisition device (video acquisition device) 220 that acquires a video visually recognized by a subject who uses the system, and a motion sickness state determination device 230.

As illustrated in FIG. 13, the biological information acquisition device 210 is attached in an oral cavity of the subject so as to be able to have contact with saliva in the oral cavity of the subject. The surrounding environment information acquisition device 220 and the motion sickness state determination device 230 are included in a display device (for example, VR viewing device or the like) 240 as its components. The display device 240 is wiredly or wirelessly connected to an information processing device (for example, game machine or the like) 250. The display device 240 may display a video (for example, VR video or the like) on the basis of data transmitted from the information processing device 250. The surrounding environment information acquisition device 220 is configured to be able to acquire a video viewed by the subject. The motion sickness state determination device 230 may be attached to the head of the subject, and preferably, attached near the ears of the subject so as to move in a same manner as the head of the subject. By being attached near the ears, an acceleration sensor 232 in the motion sickness state determination device 230 can detect a movement closer to a movement sensed by the vestibular organ of the subject.

The biological information acquisition device 210 and its components are the same as the biological information acquisition device 110 and its components described in “(2-1) Description of configuration of motion sickness state determination system of first example” in (2) described above, and the description thereof also applies to this example.

The surrounding environment information acquisition device 220 acquires video information to be presented to the subject by the display device 240.

The motion sickness state determination device 230 includes a control unit 231, the acceleration sensor 232, and a motion sickness state control unit 233. The control unit 231 includes a first index acquisition unit 234, a second index acquisition unit 235, and a motion sickness state determination unit 236.

Furthermore, the motion sickness state determination device 230 is configured to include a communication unit (not illustrated) and to be wiredly or wirelessly communicable with the biological information acquisition device 210 and the surrounding environment information acquisition device 220 using the communication unit.

The motion sickness state determination device 230 and its components are the same as the motion sickness state determination device 130 and its components described in “(2-1) Description of configuration of motion sickness state determination system of first example” in (2) described above, and the description thereof also applies to this example.

### (3-2) Description of Example of Determination Processing by Motion Sickness State Determination System of Second Example

In step S200 in FIG. 14, the motion sickness state determination system 200 starts motion sickness state determination processing. In step S200, the motion sickness state determination device 230 activates in response to a start signal of the processing input by the subject, and then, the motion sickness state determination device 230 transmits signals to activate the biological information acquisition device 210 and the surrounding environment information acquisition device 220 respectively to these devices. These devices activate in response to the receipt of the signal.

Alternatively, in response to the activation of the display device 240, all devices included in the motion sickness state determination system 200 may be activated.

Steps S201 to S205 are respectively the same as steps S101 to S105 described in “(2-2) Description of example of determination processing by motion sickness state determination system of first example” in (2) described above. Because the description applies to this example, description regarding steps S201 to S205 is omitted.

In step S211, the surrounding environment information acquisition device 220 acquires a video (for example, a game video or the like) to be presented to the subject by the display device 240. The video includes at least a part of the video presented to the subject and may include, for example, at least a video in a line-of-sight direction of the subject of the video presented to the subject. The surrounding environment information acquisition device 220 transmits the acquired video information to the motion sickness state determination device 230 through the communication unit.

In step S212, the motion sickness state determination device 230 receives the video information from the surrounding environment information acquisition device 220. The second index acquisition unit 235 of the motion sickness state determination device 230 detects an acceleration on the basis of the received video information. In the example below, the acceleration detected on the basis of the video in step S212 is referred to as a “first acceleration”. The first acceleration corresponds to the acceleration on the basis of the video viewed by the subject. The first acceleration may be an acceleration in one or two directions selected from among, for example, an acceleration in the front-back direction of the subject, an acceleration in the horizontal direction, and an acceleration in the vertical direction or may be all accelerations in these three directions.

In step S213, the acceleration sensor 232 included in the motion sickness state determination device 230 detects an acceleration. Hereinafter, the acceleration detected by the acceleration sensor in step S213 is referred to as a “second acceleration”. The second acceleration corresponds to the acceleration sensed by the vestibular organ of the subject.

In step S214, the second index acquisition unit 235 acquires the second acceleration detected by the acceleration sensor 232. The second acceleration may be one or two accelerations selected from among, for example, an acceleration in the front-back direction of the subject, an acceleration in the horizontal direction, and an acceleration in the vertical direction or may be all accelerations in these three directions. Preferably, the direction of the second acceleration may correspond to the direction of the first acceleration acquired in step S212.

In step S215, the second index acquisition unit 235 obtains a difference between the first acceleration acquired in step S212 and the second acceleration acquired in step S213. Preferably, a difference between the accelerations in the same direction is obtained. The second index acquisition unit 235 acquires the difference as the second motion sickness state index.

In step S216, the motion sickness state determination unit 236 determines whether or not the difference obtained in step S215 is equal to or more than a threshold. Hereinafter, the threshold used for the determination in step S216 is referred to as a second threshold. The second threshold may be preset or may be updated as needed. The second threshold may be set or updated on the basis of data, for example, stored in a database, more specifically, in a cloud database. As the data used for the setting or the update, for example, one or more pieces of information selected from among the difference between the first acceleration and the second acceleration acquired in the past, the motion sickness state determination result of the subject in the past, and the physical information of the subject may be used. Furthermore, as the data, a difference between a first acceleration and a second acceleration acquired in the past regarding humans other than the subject, a motion sickness state determination result of humans other than the subject in the past, physical information of humans other than the subject may be used.

In a case where the difference between the first acceleration and the second acceleration is equal to or more than the second threshold in step S216, the motion sickness state determination unit 236 proceeds the processing to step S221. In a case where the difference is less than the second threshold, the motion sickness state determination unit 236 returns the processing to steps S211 and S213, and the processing in steps S211 and S213 may be executed again.

In step S221, the motion sickness state determination unit 236 determines whether or not the variability rate or the amylase activity is equal to or more than the first threshold and the difference between the first acceleration and the second acceleration is equal to or more than the second threshold.

In a case where the variability rate or the amylase activity is equal to or more than the first threshold and the difference between the first acceleration and the second acceleration is equal to or more than the second threshold, the motion sickness state determination unit 236 determines that the subject will be in the motion sickness state or is in the motion sickness state and proceeds the processing to step S222.

In a case where the condition is not satisfied such that the variability rate or the amylase activity is equal to or more than the first threshold and the difference between the first acceleration and the second acceleration is equal to or more than the second threshold, the motion sickness state determination unit 236 determines that the subject will not be in the motion sickness state or is not in the motion sickness state and returns the processing to steps S202, S211, and S213.

In step S222, for example, the control unit 231 may select an electrode to which the current is applied and/or may select a magnitude of the current. These selections may be performed as described in “(2-2) Description of example of determination processing by motion sickness state determination system of first example” in (2) described above. Alternatively, these selections may be performed on the basis of the data stored in the display device 240 or the other components.

In step S223, the control unit 231 determines whether or not the magnitude of the current selected in step S222 exceeds an upper limit value. The upper limit value may be preset or may be appropriately changed according to the subject. The upper limit value may be, for example, three mA, four mA, or five mA.

In a case where the magnitude of the current selected in step S222 does not exceed the upper limit value, the control unit 231 proceeds the processing to step S224.

In a case where the magnitude of the current selected in step S222 exceeds the upper limit value, the control unit 231 proceeds the processing to step S225.

In step S224, the control unit 231 may drive the motion sickness state control unit 233 and apply a stimulus to the subject. In this example, the motion sickness state control unit 233 may apply, for example, a current having the magnitude selected in step S223 to the electrode selected in step S223. With this process, the current may be applied to the vestibular organ of the subject. Therefore, the difference between the first acceleration and the second acceleration can be eliminated, and it is possible to prevent to be in the motion sickness state or to suppress the motion sickness state. The application of the current may be stopped, for example, after a predetermined time has elapsed, or may be stopped in response to that dS/dt falls below the first threshold. After the stop of the current application, the processing in steps S202, S211, and S213 may be executed again.

Alternatively, while the current is continuously applied, the processing in steps S202, S211, and S213 may be executed again.

After the application of the current in step S224, the control unit 231 proceeds the processing to step S226.

A specific example of the current applied in step S224 may be as described in “(2-2) Description of example of determination processing by motion sickness state determination system of first example” in (2) described above.

In step S225, the control unit 231 may issue an error and stop the motion sickness state determination device 230. The error may be notified to the subject, for example, with sound or a video from the display device 240. Furthermore, along with the stop of the motion sickness state determination device 230, the biological information acquisition device 210 and/or the surrounding environment information acquisition device 220 may be stopped.

In step S226, the control unit 231 determines whether or not to end the motion sickness state determination processing. For example, in a case where a signal according to a subject's operation for ending the processing is detected, it is determined to end the processing. In a case where it is determined to end the processing, the control unit ends the processing. In a case where the signal is not detected, the control unit 231 returns the processing to steps S202, S211, and S213.

According to the above processing, for example, in a case where a VR video is viewed, it is possible to appropriately determine the motion sickness state. The VR video may cause the motion sickness state. Because the motion sickness state determination system in this example can appropriately determine the motion sickness state caused by the VR video as described above, for example, it is possible to notify the subject of that the subject will be in the motion sickness state or is in the motion sickness state, and it is possible to urge the subject, using the notification, to stop viewing the VR video. Furthermore, because the motion sickness state determination system in this example applies an appropriate stimulus (current) according to the acceleration difference to the subject, it is also possible to prevent to be in the motion sickness state or to suppress the motion sickness state.

(4) Third Example of First Embodiment (Application Example to VR Game Development)

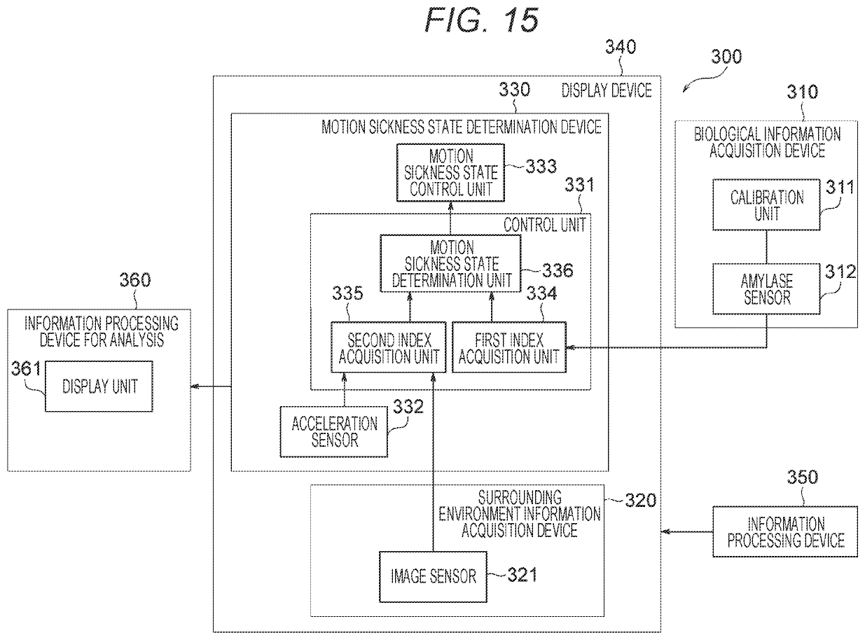

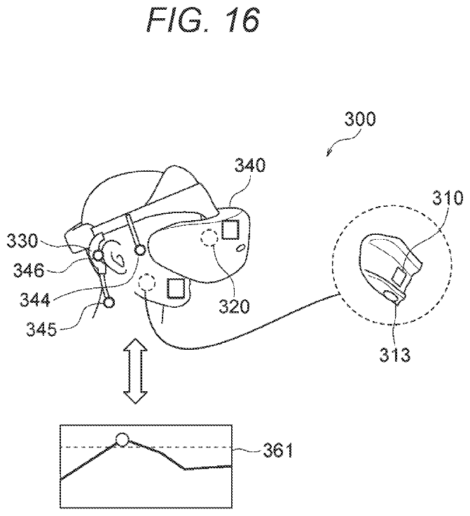

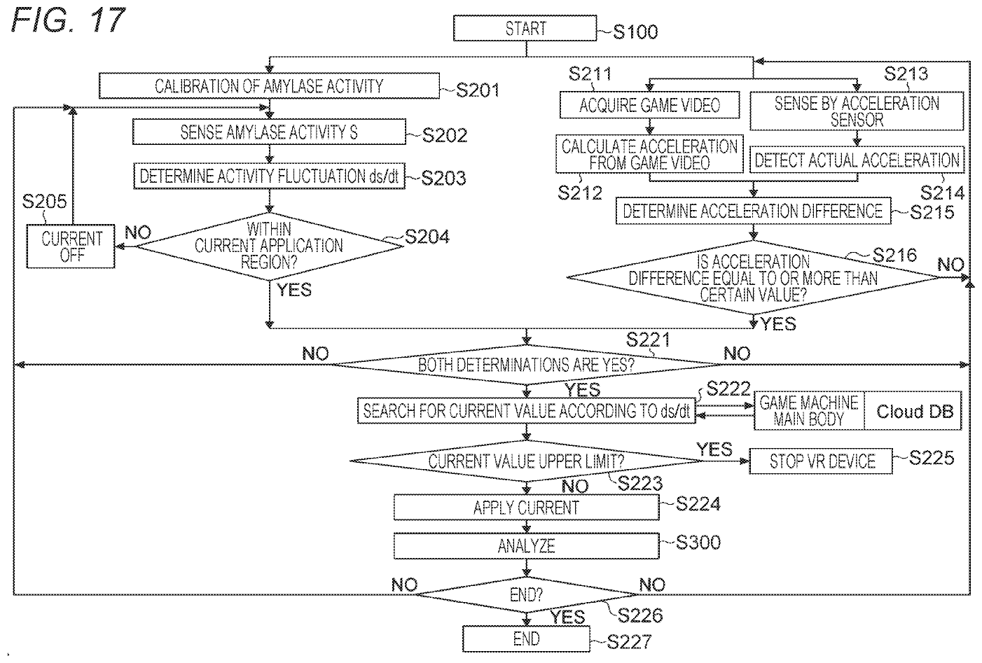

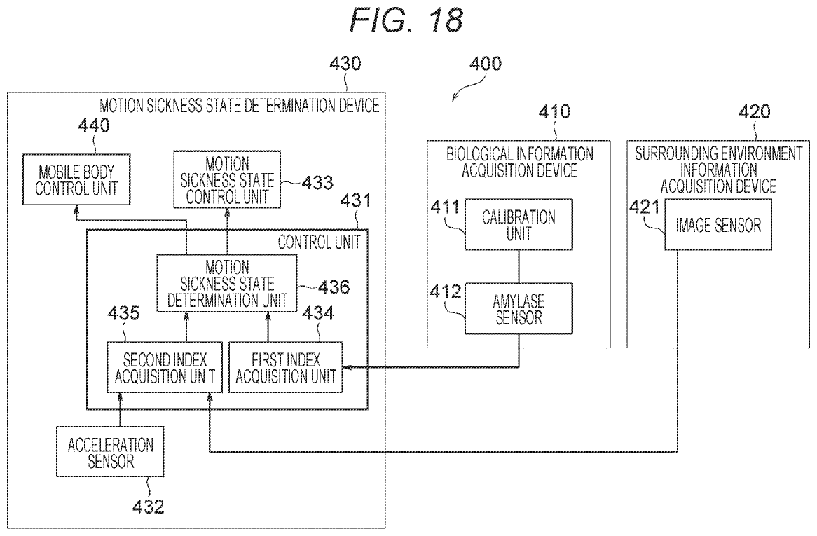

A block diagram of a motion sickness state determination system in this example is illustrated in FIG. 15, and a configuration example of the system is illustrated in FIG. 16. Furthermore, an example of a flow of motion sickness state determination processing by the system is illustrated in FIG. 17. The motion sickness state determination system in this example is a modification of the motion sickness state determination system of the second example described in (3) above for application to develop a VR game. According to the motion sickness state determination system in this example, it is possible to create a game that hardly causes a VR motion sickness more efficiently and/or in a shorter time. Moreover, it is possible to more effectively prevent the VR motion sickness.

In this example, a first index acquisition unit acquires an amylase activity in saliva as a first motion sickness state index, and a second index acquisition unit acquires a difference between accelerations acquired on the basis of movement information regarding a movement of the head of the subject and surrounding video information visually perceived by the subject as a second motion sickness state index.

(4-1) Description of Configuration of Motion Sickness State Determination System of Third Example

As illustrated in FIG. 15, a motion sickness state determination system 300 according to the present technology includes a biological information acquisition device (amylase detection device) 310 that measures an amylase activity in saliva, a surrounding environment information acquisition device (video acquisition device) 320 that acquires a video visually recognized by a subject who uses the system, and a motion sickness state determination device 330.

The biological information acquisition device 310, the surrounding environment information acquisition device 320, and the motion sickness state determination device 330 are respectively the same as the biological information acquisition device 210, the surrounding environment information acquisition device 220, and the motion sickness state determination device 230 described in “(3-1) Description of configuration of motion sickness state determination system of second example” in (3) described above, and the description thereof also applies to this example.

In this example, the surrounding environment information acquisition device 320 and the motion sickness state determination device 330 are included in a display device (for example, VR viewing device or the like) 340 as its components. The display device 340 is wiredly or wirelessly connected to an information processing device (for example, game machine or the like) 350. The display device 340 may display a video (for example, VR video or the like) on the basis of data transmitted from the information processing device 350.

Furthermore, the motion sickness state determination system 300 further includes an information processing device for analysis 360 used to analyze a motion sickness state. The information processing device for analysis 360 is wiredly or wirelessly connected to the motion sickness state determination device 330. Furthermore, the information processing device for analysis 360 may be connected to the biological information acquisition device 310 and/or the surrounding environment information acquisition device 320.

The information processing device for analysis 360 can acquire data used to determine the motion sickness state by the motion sickness state determination device 330 (for example, biological information, video information, acceleration information, or the like) and the determination result, information regarding a stimulus for control of the motion sickness state applied to the subject by the motion sickness state determination device 330 (for example, current information or the like), and information regarding an effect caused by the stimulus (for example, biological information after application of stimulus or the like) from a device included in the motion sickness state determination system 300. The information processing device for analysis 360 may analyze, for example, a relationship between a video and a motion sickness state on the basis of the acquired information. With this device, it is possible to determine whether or not the video easily causes a motion sickness state, and in addition, it is possible to improve the video so as not to cause a motion sickness state.

The information processing device for analysis 360 may include, for example, a display unit 361. The display unit 361 may display a fluctuation in biological information (for example, amylase activity or the like), for example, as illustrated in FIG. 16. Moreover, the display unit 361 may display the information regarding the stimulus for control of the motion sickness state (for example, current stimulus) applied to the subject by the motion sickness state determination device 330 and/or the acceleration information. Moreover, the display unit 361 may display video information (for example, game video or the like) presented to the subject. In this way, the information processing device for analysis 360 visualizes various types of data and presents the data to the subject. With this device, it is possible to easily determine whether or not the video easily causes a motion sickness state, and in addition, the video is easily improved so as not to cause a motion sickness state.

(4-2) Description of Example of Determination Processing by Motion Sickness State Determination System of Third Example

The flowchart illustrated in FIG. 17 is the same as the flowchart illustrated in FIG. 14 except that an analysis process in step S300 is added. Therefore, the description regarding FIG. 14 applies to steps other than step S300. Step S300 will be described below.

In step S300, the motion sickness state determination device 330 may transmit the determination result in step S221 and/or the information regarding the current applied in step S224 to the information processing device for analysis 360. Furthermore, in step S300, the motion sickness state determination device 330 may transmit data used for the determination (for example, one or more pieces of biological information, video information, and acceleration information) to the information processing device for analysis 360. The information processing device for analysis 360 may receive the data transmitted from the motion sickness state determination device 330, and then, analyze these pieces of data. Furthermore, the information processing device for analysis 360 may display these pieces of data on the display unit 361.

In FIG. 17, the transmission is performed after step S224. However, the transmission may be performed after another step. For example, after step S202, the biological information acquisition device 310 may transmit the biological information to the information processing device for analysis 360. Furthermore, after step S212, the motion sickness state determination device 230 may transmit the acceleration information to the information processing device for analysis 360. Furthermore, after step S214, the motion sickness state determination device 230 may transmit the acceleration information to the information processing device for analysis 360. A type of the information to be transmitted and a transmission timing may be appropriately selected according to required analysis.

(5) Fourth Example of First Embodiment (Application Example to Attractions in Amusement Parks)

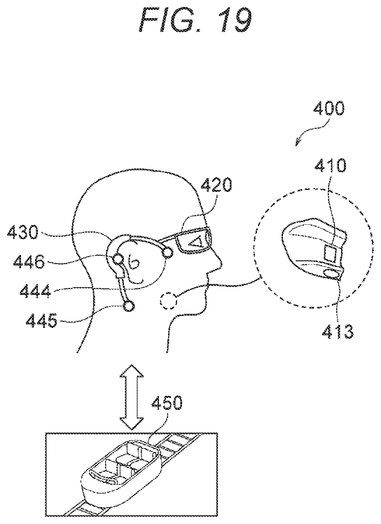

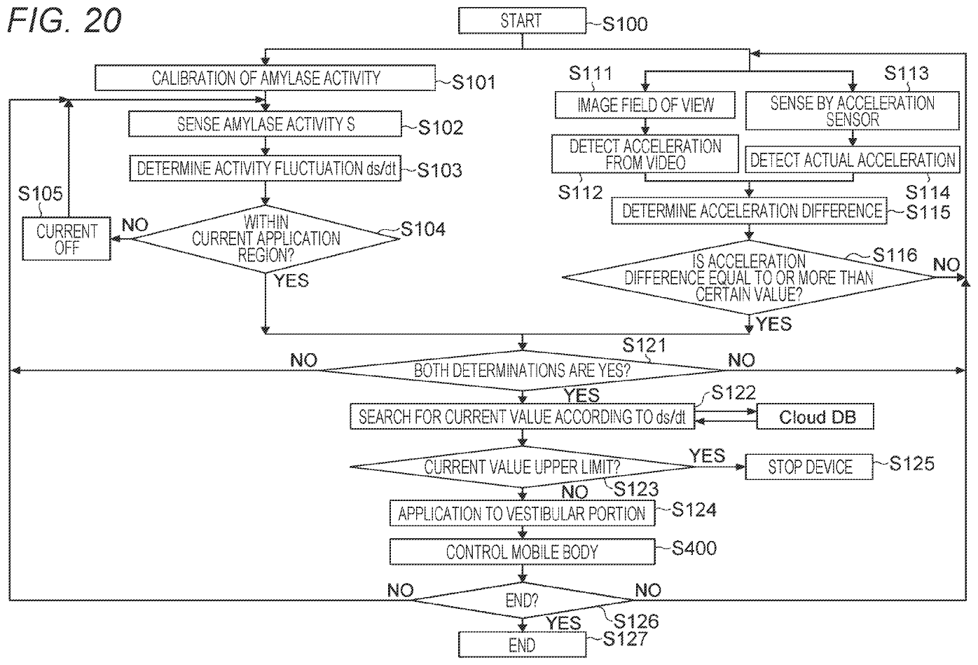

A block diagram of a motion sickness state determination system in this example is illustrated in FIG. 18, and a configuration example of the system is illustrated in FIG. 19. Furthermore, an example of a flow of motion sickness state determination processing by the system is illustrated in FIG. 20. The motion sickness state determination system in this example is a modification of the motion sickness state determination system of the first example described in (2) described above to prevent or reduce a motion sickness state of a subject who gets on an attraction of an amusement park.

In this example, a first index acquisition unit acquires an amylase activity in saliva as a first motion sickness state index, and a second index acquisition unit acquires a difference between accelerations acquired on the basis of movement information regarding a movement of the head of the subject and surrounding video information visually perceived by the subject as a second motion sickness state index.

(5-1) Description of Configuration of Motion Sickness State Determination System of Fourth Example

As illustrated in FIG. 18, a motion sickness state determination system 400 according to the present technology includes a biological information acquisition device (amylase detection device) 410 that measures an amylase activity in saliva, a surrounding environment information acquisition device (video acquisition device) 420 that acquires a video visually recognized by a subject who uses the system, and a motion sickness state determination device 430.

The biological information acquisition device 410, the surrounding environment information acquisition device 420, and the motion sickness state determination device 430 are the same as those described in “(2-1) Description of configuration of motion sickness state determination system of first example” in (2) described above, and the description thereof also applies to this example.

The motion sickness state determination system 400 further includes a mobile body control unit 440. The mobile body control unit 440 controls a movement of a mobile body on which the subject boards according to the determination result by the motion sickness state determination device 430. The mobile body control unit 440 may control, for example, an acceleration or a speed of the mobile body. The mobile body may be an attraction 450 in an amusement park, for example, as illustrated in FIG. 19. Alternatively, the mobile body may be a vehicle other than the attraction. The mobile body may be, for example, a vehicle such as an automobile.

Similarly to the control unit 11 described in 1. above, the mobile body control unit 440 may include a hard disk, a CPU or MPU, and a memory that store programs and an OS used to make the motion sickness state determination device 430 control the mobile body. The mobile body control unit 440 may be included in a control unit 431.

By controlling the movement of the mobile body by the mobile body control unit 440, it is possible to prevent the subject from being in the motion sickness state or to relieve the motion sickness state. The prevention or the relief of the motion sickness state is performed by controlling the mobile body, for example, so as to reduce the difference between the first acceleration and the second acceleration.

Because the motion sickness state can be prevented or relieved by controlling the mobile body by the mobile body control unit 440, for example, it is possible to reduce a degree of a stimulus applied by a motion sickness state control unit. Alternatively, it is possible to prevent or relieve the motion sickness state without applying the stimulus by the motion sickness state control unit.

(5-2) Description of Example of Determination Processing by Motion Sickness State Determination System of Fourth Example

The flowchart illustrated in FIG. 20 is the same as the flowchart illustrated in FIG. 7 except that step 400 is added. Therefore, the description regarding FIG. 7 applies to steps other than step S400. Step S400 will be mainly described below.

In step S400, the mobile body control unit 440 controls the movement of the mobile body. For example, the mobile body control unit 440 may decrease or increase the acceleration or the speed of the mobile body so as to reduce the difference between the first acceleration and the second acceleration. With this unit, it is possible to prevent the subject from being in the motion sickness state or to relieve the motion sickness state of the subject.

In the flowchart illustrated in FIG. 20, the motion sickness state is prevented or relieved by controlling the movement of the mobile body in step S400. Therefore, a current applied in step S224 may be smaller than that in a case where step S400 is not performed or it is not necessary to perform steps S222, S223, and S224. For example, in a case where the motion sickness state determination unit 236 determines in step S221 that the subject will be in the motion sickness state or is in the motion sickness state, the control unit 231 proceeds the processing to step S400.

(6) Fifth Example of First Embodiment (Application Example to Training Simulators)

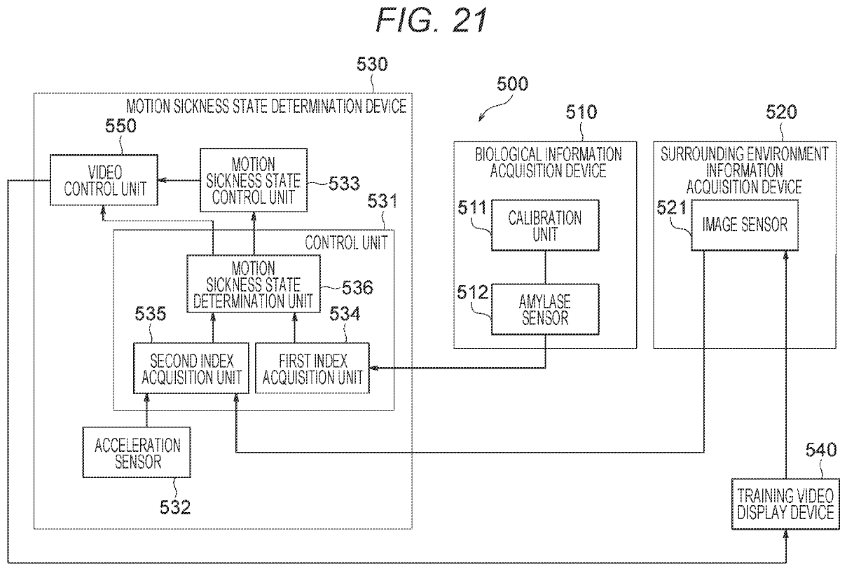

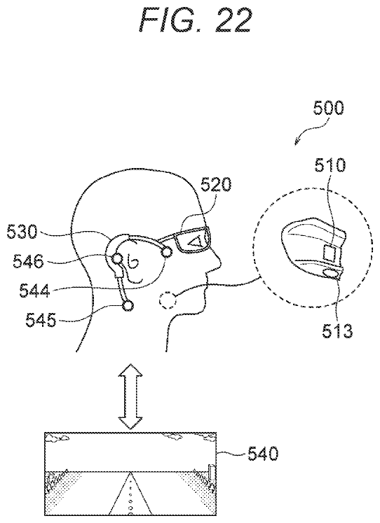

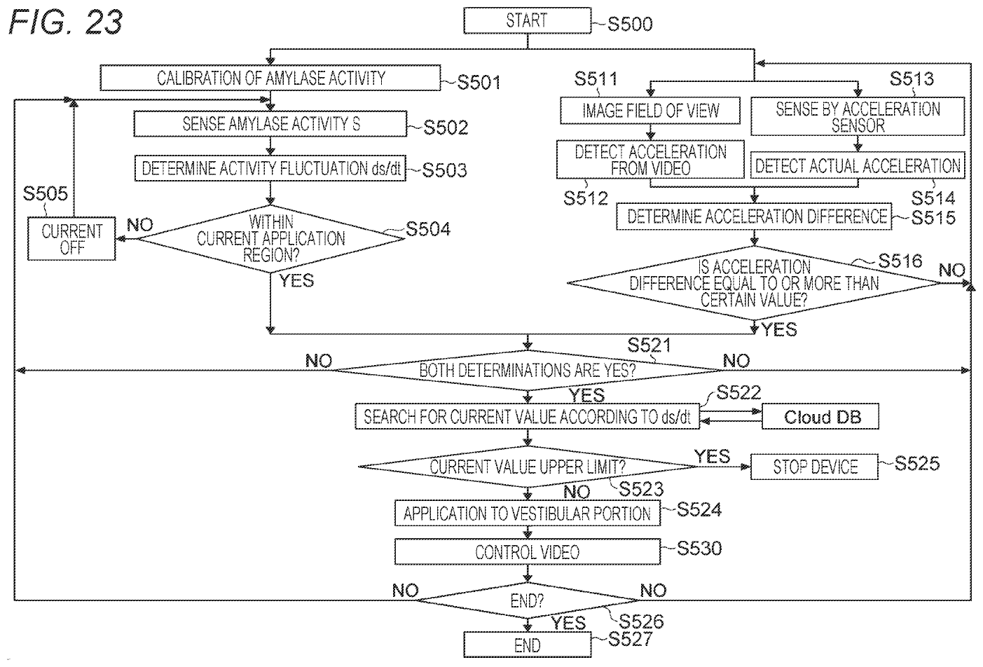

A block diagram of a motion sickness state determination system in this example is illustrated in FIG. 21, and a configuration example of the system is illustrated in FIG. 22. Furthermore, an example of a flow of motion sickness state determination processing by the system is illustrated in FIG. 23. The motion sickness state determination system in this example configures the motion sickness state determination system of the first example described in (2) above as a training simulator that makes an occupant of a mobile body (for example, ship, airplane, or the like) be less likely to feel a motion sickness.

In this example, a first index acquisition unit acquires an amylase activity in saliva as a first motion sickness state index, and a second index acquisition unit acquires a difference between accelerations acquired on the basis of movement information regarding a movement of the head of the subject and surrounding video information visually perceived by the subject as a second motion sickness state index.

(6-1) Description of Configuration of Motion Sickness State Determination System of Fifth Example

As illustrated in FIG. 21, a motion sickness state determination system 500 according to the present technology includes a biological information acquisition device (amylase detection device) 510 that measures an amylase activity in saliva, a surrounding environment information acquisition device (video acquisition device) 520 that acquires a video visually recognized by a subject who uses the system, and a motion sickness state determination device 530.

The biological information acquisition device 510 and the motion sickness state determination device 530 are the same as those described in “(2-1) Description of configuration of motion sickness state determination system of first example” in (2) described above, and the description thereof also applies to this example.

The motion sickness state determination system 500 may be used in combination with a training video display device 540. As illustrated in FIG. 22, the training video display device 540 presents a video and/or a movement (for example, vibration or the like) as if the subject is on a mobile body, for example, an airplane, a ship, or the like that easily causes a motion sickness to the subject.

The surrounding environment information acquisition device 520 acquires, for example, video information to be presented to the subject by the training video display device 540. The surrounding environment information acquisition device 520 includes, for example, an image sensor 521. The surrounding environment information acquisition device 520 is configured to further include a communication unit (not illustrated) and to be wiredly or wirelessly communicable with the motion sickness state determination device 530 using the communication unit. The surrounding environment information acquisition device 520 may be configured as an eyewear as illustrated in FIG. 22. The image sensor 521 acquires a video similar to an external landscape visually perceived by the subject who wears the eyewear. The image sensor 521 may be, for example, a CMOS or a CCD.

A motion sickness state control unit 533 included the motion sickness state determination system 500 may apply, for example, a current that causes a difference between the first acceleration and the second acceleration or increase the difference between the first acceleration and the second acceleration to the vestibular organ of the subject. As a result, for example, an environment where the motion sickness easily occurs can be simulated, and it is possible to perform training against the motion sickness.

The motion sickness state determination system 500 may include a video control unit 550 that controls a video displayed on the training video display device 540. The video control unit 550 may control the video displayed on the training video display device 540, for example, according to the current applied by the motion sickness state control unit 533. For example, the video may be controlled so as to easily cause a motion sickness.

Furthermore, the motion sickness state determination system 500 may include, for example, a movement control unit (not illustrated) that moves a seat or the like where a training target sits so as to apply a movement (for example, vibration) to the training target. By applying the movement to the training target by the movement control unit, a motion sickness may more easily occur.

(6-2) Description of Example of Determination Processing by Motion Sickness State Determination System of Fifth Example

The flowchart in FIG. 23 mainly differs from the flowchart in FIG. 7 in that step S530 is added. Furthermore, other steps including a step for acquiring a first acceleration also differs. The differences from the flowchart illustrated in FIG. 7 will be mainly described below.

In step S500 in FIG. 23, the motion sickness state determination system 500 starts motion sickness state determination processing in response to activation of the training video display device 540. More specifically, the motion sickness state determination device 530, the biological information acquisition device 510, and the surrounding environment information acquisition device 520 included in the motion sickness state determination system 500 are activated.

Steps S501 to S505 are respectively the same as steps S101 to S105 described in “(2-2) Description of example of determination processing by motion sickness state determination system of first example” in (2) described above. Because the description applies to this example, description regarding steps S501 to S505 is omitted.

In step S511, the surrounding environment information acquisition device 520 acquires video information to be presented to the subject from the training video display device 540. The video includes at least a part of a video viewed by the subject and may include, for example, at least a video in the line-of-sight direction of the subject. More specifically, the image sensor 521 may acquire the video. The surrounding environment information acquisition device 520 transmits the acquired video information to the motion sickness state determination device 530 through the communication unit.

In step S512, the motion sickness state determination device 530 receives the video information from the surrounding environment information acquisition device 520. A second index acquisition unit 535 of the motion sickness state determination device 530 detects an acceleration on the basis of the received video information. Hereinafter, the acceleration detected on the basis of the video in step S512 is referred to as a “first acceleration”. The first acceleration corresponds to the acceleration on the basis of the video viewed by the subject. The first acceleration may be one or two accelerations selected from among, for example, an acceleration in the front-back direction of the subject, an acceleration in the horizontal direction, and an acceleration in the vertical direction or may be all accelerations in these three directions.

Steps S513 to S516 and S521 are respectively the same as steps S113 to S116 and S121 described in “(2-2) Description of example of determination processing by motion sickness state determination system of first example” in (2) described above. Because the description applies to this example, description regarding steps S513 to S516 and S521 is omitted.

In step S522, for example, a control unit 531 may select an electrode to which a current is applied and/or may select a magnitude of the current.

The electrode to which the current is applied may be appropriately selected according to a direction in which it is desired to make the subject feel the acceleration. As described above, to make the subject feel an acceleration difference in the vertical direction, the electrode near the mastoid and the electrode in the neck portion may be selected as the electrodes to which the current is applied. To make the subject feel an acceleration difference in the horizontal direction, the electrodes near the left and right temples are selected as the electrodes to which the current is applied. To make the subject feel an acceleration difference in the front-back direction, the electrode near the mastoid and the electrodes near the temples are selected as the electrodes to which the current is applied.

The magnitude of the current to be applied may be set on the basis of, for example, a relationship between an applied current and a motion sickness state triggered on the basis of the current. In the present technology, the magnitude of the current to be applied may be, for example, 0.01 mA to three mA, more preferably, 0.1 mA to two mM, even more preferably, 0.5 mA to 1.5 mA.

The applied current is not gradually increased to the selected value, and the current of the selected value may be applied from the start of the application of the current. This makes it possible to make the subject feel the acceleration that triggers the motion sickness state.

In step S523, the control unit 531 determines whether or not the magnitude of the current selected in step S522 exceeds an upper limit value. The upper limit value may be preset or may be appropriately changed according to the subject. The upper limit value may be, for example, three mA, four mA, or five mA.

In a case where the magnitude of the current selected in step S522 does not exceed the upper limit value, the control unit 531 proceeds the processing to step S524.

In a case where the magnitude of the current selected in step S522 exceeds the upper limit value, the control unit 531 proceeds the processing to step S525.

In step S525, the control unit 531 may issue an error and stop the training video display device 540 and the motion sickness state determination device 530. The error may be notified to the subject, for example, with sound or a video. Furthermore, along with the stop of the motion sickness state determination device 530, the biological information acquisition device 510 and/or the surrounding environment information acquisition device 520 may be stopped.

In step S524, the control unit 531 may drive the motion sickness state control unit 533 and apply a stimulus to the subject. In this example, for example, the current having the magnitude selected in step S523 may be applied to the electrode selected in step S523. This makes it possible to make the subject feel the acceleration that triggers the motion sickness state. The application of the current may be stopped, for example, after a predetermined time has elapsed. After the application of the current in step S524, the control unit 531 proceeds the processing to step S530.

In step S530, the video control unit 550 may control the video displayed on the training video display device 540, for example, according to the current applied by the motion sickness state control unit 533 in step S524. For example, the video may be controlled so as to easily cause a motion sickness.

Note that, step S530 may be performed after (particularly, immediately after) step S524 or may be performed at the same time as step S524.

In step S526, the control unit 531 determines whether or not to end the motion sickness state determination processing. For example, in a case where a signal according to a subject's operation for ending the processing is detected, it is determined to end the processing. In a case where it is determined to end the processing, the control unit 531 ends the processing. In a case where the signal is not detected, the control unit 531 returns the processing to steps S502, S511, and S513.

According to the above processing, for example, it is possible to make the subject feel the acceleration that triggers the motion sickness state, and this is useful for making the subject to have resistant to the acceleration that may cause the motion sickness state.

(7) Sixth Example of First Embodiment (Example of Determination Based on Amylase Activity and Odor)

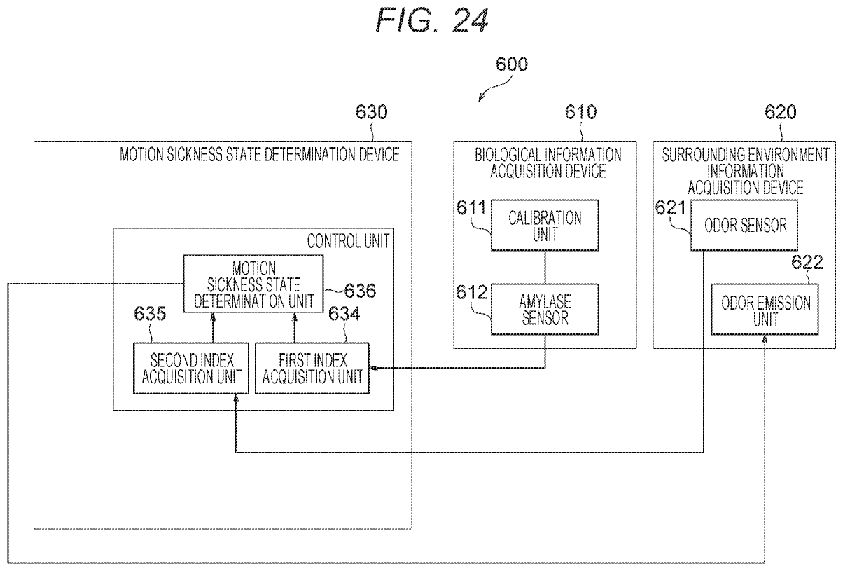

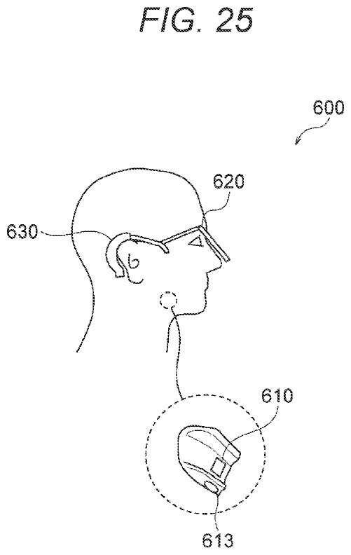

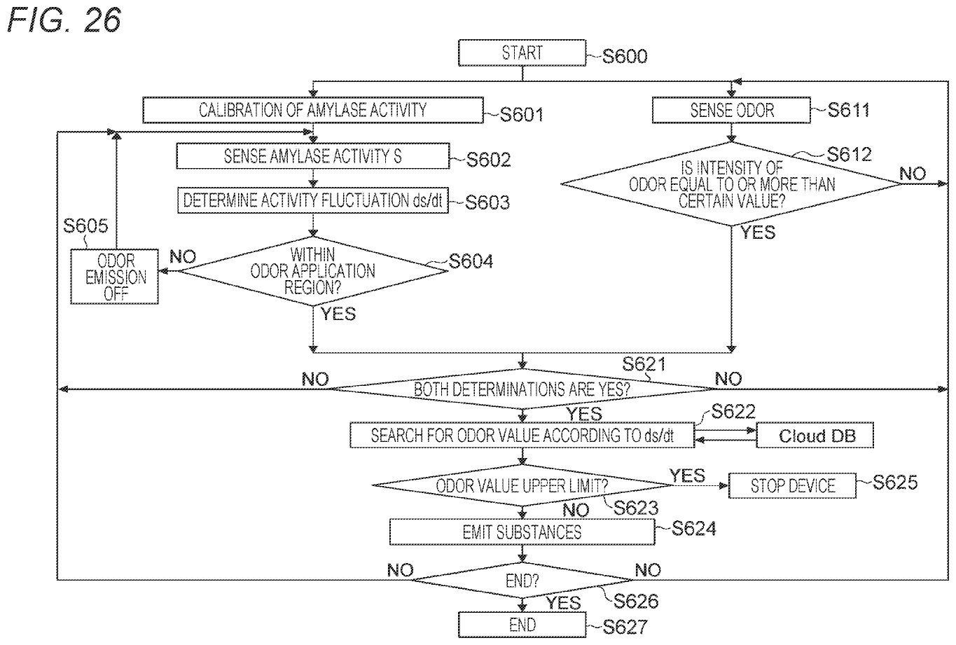

A block diagram of a motion sickness state determination system in this example is illustrated in FIG. 24, and a configuration example of the motion sickness state determination system in this example is illustrated in FIG. 25. Furthermore, an example of a flow of motion sickness state determination processing by the system is illustrated in FIG. 26. In this example, a first index acquisition unit acquires an amylase activity in saliva as a first motion sickness state index, and a second index acquisition unit acquires odor information regarding an odor perceived by sense of smell by a subject as a second motion sickness state index.

(7-1) Description of Configuration of Motion Sickness State Determination System of Sixth Example

As illustrated in FIG. 24, a motion sickness state determination system 600 according to the present technology includes a biological information acquisition device (amylase detection device) 610 that measures an amylase activity in saliva, a surrounding environment information acquisition device (odor detection device) 620 that acquires odor information perceived by sense of smell by a subject who uses the system, and a motion sickness state determination device 630.

As illustrated in FIG. 25, the biological information acquisition device 610 is attached in an oral cavity of the subject so as to be able to have contact with saliva in the oral cavity of the subject. The surrounding environment information acquisition device 620 is attached near the nose of the subject so as to acquire information regarding an odor felt by the subject. The motion sickness state determination device 630 may be attached to the head of the subject and, for example, may be attached near the ear of the subject.

The biological information acquisition device 610 is the same as the biological information acquisition device 110 described in “(2-1) Description of configuration of motion sickness state determination system of first example” in (2) described above, and the description thereof also applies to this example.

The surrounding environment information acquisition device 620 includes an odor sensor 621 and a motion sickness state control unit (odor emission unit) 622. The surrounding environment information acquisition device 620 is configured to further include a communication unit (not illustrated) and to be wiredly or wirelessly communicable with the motion sickness state determination device 630 using the communication unit.

The surrounding environment information acquisition device 620 may be configured so that the odor sensor 621 is disposed near the nose. With this configuration, the odor sensor 621 can detect the odor closer to the odor sensed by the subject.

The odor sensor 621 may be any odor sensor selected from among, for example, a conductometric type odor sensor (chemoresistor), a capacitive type odor sensor (chemocapacitor), a potentiometric type odor sensor (chemodiode, chemotransistor, or the like), a calorimetric type odor sensor (thermo-chemosensor), a gravimetric type odor sensor, an optical type odor sensor, and an electrochemical type odor sensor.

The motion sickness state control unit (odor emission unit) 622 presents the odor to the subject or removes the odor around the subject on the basis of the determination result of a motion sickness state determination unit 636. More specifically, the motion sickness state control unit (odor emission unit) 622 emits substances that prevent the subject from being in the motion sickness state or relieve the motion sickness state according to the result of the determination by the motion sickness state determination unit 636. The substance may be, for example, a substance that eliminates or masks the odor that may cause the motion sickness state or may be a substance having an odor stronger than the odor.

The motion sickness state determination device 630 includes a control unit 631, and the control unit 631 includes a first index acquisition unit 634, a second index acquisition unit 635, and a motion sickness state determination unit 636.

Furthermore, the motion sickness state determination device 130 is configured to include a communication unit (not illustrated) and to be wiredly or wirelessly communicable with the biological information acquisition device 610 and the surrounding environment information acquisition device 620.

The first index acquisition unit 634 acquires the amylase activity acquired by the amylase sensor 612 as a first motion sickness state index.

The second index acquisition unit 635 acquires the odor information acquired by the odor sensor 621 of the surrounding environment information acquisition device 620 as a second motion sickness state index.

The motion sickness state determination unit 636 determines whether or not the subject will be in the motion sickness state or is in the motion sickness state on the basis of the first motion sickness state index and the second motion sickness state index.

(7-2) Description of Example of Determination Processing by Motion Sickness State Determination System of Sixth Example

In step S600 in FIG. 26, the motion sickness state determination system 600 starts motion sickness state determination processing. In step S600, the motion sickness state determination device 630 activates in response to a start signal of the processing input by the subject, and then, the motion sickness state determination device 630 transmits signals to activate the biological information acquisition device 610 and the surrounding environment information acquisition device 620 respectively to these devices. These devices activate in response to the receipt of the signal.

Steps S601 to S603 are respectively the same as steps S101 to S103 described in “(2-2) Description of example of determination processing by motion sickness state determination system of first example” in (2) described above. Because the description applies to this example, description regarding steps S601 to S603 is omitted.

In step S604, the control unit 631 (for example, motion sickness state determination unit 636) determines whether or not the amylase activity or the variability rate is equal to or more than a threshold. Hereinafter, the threshold used for the determination in step S604 is referred to as a first threshold. The first threshold may be preset or may be updated as needed. The first threshold may be set or updated on the basis of data, for example, stored in a database, more specifically, in a cloud database. As the data used for the setting or update, for example, one or more pieces of information selected from among amylase activity information of the subject measured in the past, motion sickness state determination results of the subject in the past, physical information of the subject may be used. Furthermore, as the data, amylase activity information of humans other than the subject, motion sickness state determination results of humans other than the subject in the past, and physical information of humans other than the subject may be used.

In step S604, in a case where the amylase activity or the variability rate is equal to or more than the first threshold, the motion sickness state determination unit 636 proceeds the processing to step S605. In a case where the amylase activity or the variability rate is less than the threshold, the motion sickness state determination unit 636 proceeds the processing to step S621.

In step S605, the processing returns to step S602, and the processing in steps S602 to S604 may be executed again.

Furthermore, in a case where steps S602 to S604 are performed in a state where the motion sickness state control unit 633 presents the odor to the subject, the odor emission unit 633 stops the presentation of the odor in step S605.

In step S611, the surrounding environment information acquisition device 620 detects an odor near the nose of the subject. The odor may be detected, more specifically, by the odor sensor 621. The surrounding environment information acquisition device 620 transmits information regarding the detected odor to the motion sickness state determination device 630 through the communication unit. The information regarding the odor may be more preferably an intensity of a predetermined type of odor.

In step S612, the motion sickness state determination device 630 receives the information regarding the odor from the surrounding environment information acquisition device 620. The second index acquisition unit 635 of the motion sickness state determination device 630 acquires the received information regarding the odor as a second motion sickness state index.

In step S612, the motion sickness state determination unit 636 determines whether or not the second motion sickness state index (for example, intensity of odor or the like) acquired in step S115 described above is equal to or more than a threshold. Hereinafter, the threshold used for the determination in step S612 is referred to as a second threshold. The second threshold may be preset or may be updated as needed. The second threshold may be set or updated on the basis of data, for example, stored in a database, more specifically, in a cloud database. As the data used for the setting or update, for example, one or more pieces of information selected from among odor information acquired in the past, motion sickness state determination results of the subject in the past, physical information of the subject may be used. Furthermore, as the data, odor information acquired in the past regarding humans other than the subject, motion sickness state determination results of humans other than the subject in the past, physical information of humans other than the subject may be used.

In a case where the second motion sickness state index is equal to or more than the second threshold in step S612, the motion sickness state determination unit 636 proceeds the processing to step S621. In a case where the second motion sickness state index is less than the second threshold, the processing is returned to step S611, and steps S611 and S612 may be performed again.

In step S621, the motion sickness state determination unit 636 determines whether or not the variability rate or the amylase activity is equal to or more than the first threshold and the second motion sickness state index is equal to or more than the second threshold.

In a case where the variability rate or the amylase activity is equal to or more than the first threshold and the second motion sickness state index is equal to or more than the second threshold, the motion sickness state determination unit 636 determines that the subject will be in the motion sickness state or is in the motion sickness state and proceeds the processing to step S622.

In a case where the condition is not satisfied such that the amylase activity or the variability rate is equal to or more than the first threshold and the second motion sickness state index is equal to or more than the second threshold, the motion sickness state determination unit 636 determines that the subject will not be in the motion sickness state or is not in the motion sickness state and returns the processing to steps S602 and S611.

In step S622, for example, the control unit 631 may select the type of the odor to be presented to the subject and/or the intensity of the odor to be presented to the subject.

The type of the odor may be selected according to the type of the odor detected by the odor sensor. The intensity of the odor may be selected, for example, on the basis of the variability rate dS/dt of the amylase activity S or the amylase activity S. For example, the intensity of the odor may be dS/dt×C. C is a correction item and, for example, may be a preset value or may be derived on the basis of the data in the cloud database.

In step S622, the cloud database may be referred, and the type of the odor to be presented and/or the intensity of the odor to be presented may be retrieved from the cloud database. The intensity of the odor may be, more specifically, an emission amount of an odor substance. For example, the cloud database may include one or more type of data selected from among physical information of the subject (for example, gender, age, height, weight, or the like), motion sickness state determination results of the subject in the past, an odor presented history, and the presented odor and a response of the amylase activity caused by the odor. The cloud database may include one or more types of data selected from among physical information of each of humans other than the subject (for example, gender, age, height, weight, or the like), motion sickness state determination results of humans other than the subject in the past, an odor presented history, and the presented odor and a response of the amylase activity caused by the odor.

For example, in a case where the subject uses the motion sickness state determination system in this example for the first time, motion sickness state determination result data regarding the subject is not accumulated. Therefore, a database regarding humans other than the subject may be used. For example, from data regarding another person who has physical information closest to the physical information of the subject or data regarding a plurality of other persons having physical information close to the physical information of the subject, the type of the odor to be presented and/or the intensity of the odor may be selected.

In step S623, the control unit 631 determines whether or not the odor intensity selected in step S622 exceeds an upper limit value. The upper limit value may be preset or may be appropriately changed according to the subject.

In a case where the odor intensity selected in step S622 does not exceed the upper limit value, the control unit 631 proceeds the processing to step S624.

In a case where the odor intensity selected in step S622 exceeds the upper limit value, the control unit 631 proceeds the processing to step S625.

In step S625, the control unit 631 may issue an error and stop the motion sickness state determination device 630. The error may be notified to the subject, for example, with sound or a video. Furthermore, along with the stop of the motion sickness state determination device 630, the biological information acquisition device 610 and/or the surrounding environment information acquisition device 620 may be stopped.

In step S624, the control unit 631 may drive the odor emission unit 623 and apply a stimulus to the subject. In this example, for example, the odor of which the type and the intensity are selected in step S622 may be presented to the subject. With this process, an effect of the odor that may cause or has caused the motion sickness state on the subject can be eliminated or reduced, and it is possible to prevent the subject from being in the motion sickness state or to suppress the motion sickness state. The presentation of the odor may be stopped, for example, after a predetermined time has elapsed, or may be stopped in response to that dS/dt falls below the first threshold. After the presentation of the odor, the processing in steps S602 and S611 may be executed again.

Alternatively, while the odor is presented, the processing in steps S602 and S611 may be executed again.

After the application of the current in step S624, the control unit 631 proceeds the processing to step S626.

In step S626, the control unit 631 determines whether or not to end the motion sickness state determination processing. For example, in a case where a signal according to a subject's operation for ending the processing is detected, it is determined to end the processing. In a case where it is determined to end the processing, the control unit ends the processing. In a case where the signal is not detected, the control unit 631 returns the processing to steps S602 and S611.

According to the above processing, for example, it is possible to appropriately determine the motion sickness state of the subject who feels an unpleasant odor. Because the motion sickness state determination system in this example can appropriately determine the motion sickness state as described above, for example, it is also possible to notify the subject of that the subject will be in the motion sickness state or is in the motion sickness state, and it is possible to urge the subject to perform ventilation to eliminate the odor in response to the notification. Furthermore, because the motion sickness state determination system in this example can present the odor that eliminates the unpleasant odor to the subject, it is possible to prevent the subject from being in the motion sickness state or to suppress the motion sickness state.

### 2. Second Embodiment (Biological Information Acquisition Device)

The present technology provides a biological information acquisition device that includes a biological information acquisition unit that acquires biological information of a subject and is used in combination with an index acquisition device that acquires a motion sickness state index on the basis of movement information regarding a movement of the subject and/or surrounding environment information perceived by the subject in order to determine a motion sickness state of the subject.

The biological information acquisition device is the biological information acquisition device described in 1. above, and the description thereof applies to the present embodiment. The biological information acquisition unit included in the device may be, for example, the enzyme sensor or the odor sensor described in 1. above.

The index acquisition device used in combination with the biological information acquisition device may be the motion sickness state determination device as described in 1. above. The combination of these devices can appropriately determine the motion sickness state.

### 3. Third Embodiment (Surrounding Environment Information Acquisition Device)

The present technology provides a surrounding environment information acquisition device that includes a surrounding environment information acquisition unit that acquires surrounding environment information perceived by a subject and is used in combination with a biological information acquisition device that acquires biological information of the subject as a motion sickness state index in order to determine a motion sickness state of the subject. The surrounding environment information acquisition device is the surrounding environment information acquisition device described in 1. above, and the description thereof applies to the present embodiment.

The biological information acquisition device used in combination with the surrounding environment information acquisition device may be the biological information acquisition device as described in 1. above. By using this combination in combination with the motion sickness state determination device as described in 1. above, it is possible to appropriately determine the motion sickness state.

### 4. Fourth Embodiment (Motion Sickness State Determination Device)

The present technology provides a motion sickness state determination device that includes a first index acquisition unit that acquires biological information of a subject as a first motion sickness state index, a second index acquisition unit that acquires a second motion sickness state index on the basis of movement information regarding a movement of the subject and/or surrounding environment information perceived by the subject, and a motion sickness state determination unit that determines whether or not the subject will be in a motion sickness state or is in the motion sickness state on the basis of the first motion sickness state index and the second motion sickness state index. The motion sickness state determination device is the motion sickness state determination device described in 1. above, and the description thereof applies to the present embodiment.

### 5. Fifth Embodiment (Motion Sickness State Determination Method)

A motion sickness state determination method according to the present technology includes a first index acquisition process for acquiring biological information of a subject as a first motion sickness state index, a second index acquisition process for acquiring a second motion sickness state index on the basis of movement information regarding a movement of the subject and/or surrounding environment information perceived by the subject, and a motion sickness state determination process for determining whether or not the subject will be in a motion sickness state or is in the motion sickness state on the basis of the first motion sickness state index and the second motion sickness state index. According to the motion sickness state determination method according to the present technology, the effect described in (1-2) in 1. above is achieved.

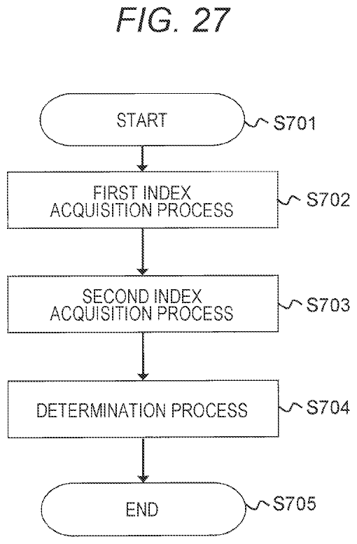

Hereinafter, each step will be described with reference to FIGS. 3 and 27. FIG. 3 is as described in 1. above. FIG. 27 is an example of a flowchart of the motion sickness state determination method.

In step S701 in FIG. 27, a motion sickness state determination system 1 starts video projection processing according to the present technology.

In the first index acquisition process in step S702, a first index acquisition unit 12 acquires the biological information of the subject as the first motion sickness state index. The biological information and the first motion sickness state index are as described in (1-2) in 1. above, and the description thereof applies to the present embodiment. Furthermore, this step corresponds to, for example, step S103 described in (2-2) in 1. above, and the description thereof applies to this step.

In the second index acquisition process in step S703, a second index acquisition unit 13 acquires the second motion sickness state index on the basis of the movement information regarding the movement of the subject and/or the surrounding environment information perceived by the subject. The movement information, the surrounding environment information, and the second motion sickness state index are as described in (1-2) in 1. above, and the description thereof applies to the present embodiment. This step corresponds to, for example, step S115 described in (2-2) in 1. above, and the description thereof applies to this step.

Steps S702 and S703 may be performed in this order or in the reverse order or may be concurrently performed.

In the determination process in step S704, a motion sickness state determination unit 14 determines whether or not the subject will be in the motion sickness state or is in the motion sickness state on the basis of the first motion sickness state index and the second motion sickness state index. Details of the determination are as described in (1-2) in 1. above, and the description thereof applies to the present embodiment. This step corresponds to, for example, step S121 described in (2-2) in 1. above, and the description thereof applies to this step.

In step S705, the motion sickness state determination system 1 ends the motion sickness state determination processing according to the present technology.

Steps S702 to S704 may be more preferably repeated.

Between steps S704 and S705, a motion sickness state control process may be performed in which a motion sickness state control unit 15 controls the motion sickness state of the subject on the basis of the determination result in step S704. The motion sickness state control process corresponds to, for example, step S124 described in (2-2) in 1. above, and the description thereof applies to this step. After the motion sickness state control process, steps S702 to S704 may be repeated again.

A more detailed specific example of the motion sickness state determination method according to the present technology is as described in each example in 1. above, and the description thereof applies to the embodiment. For example, steps described in these examples may be performed in the motion sickness state determination method according to the present technology.

Note that the present technology can have the following configuration.

[1] A motion sickness state determination system including:

a first index acquisition unit configured to acquire biological information of a subject as a first motion sickness state index;

a second index acquisition unit configured to acquire a second motion sickness state index on the basis of movement information regarding a movement of the subject and/or surrounding environment information perceived by the subject; and

a motion sickness state determination unit configured to determine whether or not the subject will be in a motion sickness state or is in the motion sickness state on the basis of the first motion sickness state index and the second motion sickness state index.

[2] The motion sickness state determination system according to [1], further including: a motion sickness state control unit configured to control the motion sickness state of the subject on the basis of a determination result of the motion sickness state determination unit.

[3] The motion sickness state determination system according to [2], in which the motion sickness state control unit determines processing content used to control the motion sickness state on the basis of information acquired from a database.

[4] The motion sickness state determination system according to any one of [1] to [3], in which the motion sickness state of the subject is determined in real time.

[5] The motion sickness state determination system according to any one of [1] to [4], in which in a case where both of the first motion sickness state index and the second motion sickness state index satisfy references set for the respective indexes, the motion sickness state determination unit determines that the subject will be in the motion sickness state or is in the motion sickness state.

[6] The motion sickness state determination system according to any one of [1] to [5], in which the biological information includes information regarding an enzyme derived from the subject.

[7] The motion sickness state determination system according to [6], in which the enzyme includes an amylase.

[8] The motion sickness state determination system according to any one of [1] to [7], in which

the movement information is movement information regarding a movement of a body of the subject,

the surrounding environment information is video information regarding a video visually perceived by the subject, and

the second index acquisition unit acquires a second motion sickness state index on the basis of the movement information and the video information.

[9] The motion sickness state determination system according to [8], in which

the movement information is first acceleration information based on the movement of the body of the subject,

the surrounding environment information is second acceleration information based on a video visually perceived by the subject, and

the second index acquisition unit acquires a second motion sickness state index on the basis of the first acceleration information and the second acceleration information.

[10] The motion sickness state determination system according to [9], in which the second index acquisition unit acquires a difference between the first acceleration information and the second acceleration information as the second motion sickness state index.

[11] The motion sickness state determination system according to any one of [8] to [10], further including: a motion sickness state control unit configured to apply a current to a head of the subject on the basis of a determination result of the motion sickness state determination unit.

[12] The motion sickness state determination system according to any one of [1] to [7], in which

the surrounding environment information is odor information regarding an odor perceived by sense of smell by the subject, and

the second index acquisition unit acquires a second motion sickness state index on the basis of the odor information.

[13] The motion sickness state determination system according to [12], in which the second index acquisition unit acquires an intensity of a predetermined type of odor as the second motion sickness state index.

[14] The motion sickness state determination system according to [12] or [13], further including: a motion sickness state control unit configured to present an odor to the subject or remove an odor around the subject on the basis of a determination result of the motion sickness state determination unit.

[15] The motion sickness state determination system according to any one of [1] to [14], further including: an output unit configured to output the determination result of the motion sickness state determination unit.

[16] A biological information acquisition device including:

a biological information acquisition unit configured to acquire biological information of a subject, in which

the biological information acquisition device is used in combination with an index acquisition device that acquires a motion sickness state index on the basis of movement information regarding a movement of the subject and/or surrounding environment information perceived by the subject in order to determine a motion sickness state of the subject.

[17] A surrounding environment information acquisition device including:

a surrounding environment information acquisition unit configured to acquire surrounding environment information perceived by a subject, in which

the surrounding environment information acquisition device is used in combination with a biological information acquisition device that acquires biological information of the subject as a motion sickness state index in order to determine a motion sickness state of the subject.

[18] A motion sickness state determination device including:

a first index acquisition unit configured to acquire biological information of a subject as a first motion sickness state index;

a second index acquisition unit configured to acquire a second motion sickness state index on the basis of movement information regarding a movement of the subject and/or surrounding environment information perceived by the subject; and

a motion sickness state determination unit configured to determine whether or not the subject will be in a motion sickness state or is in the motion sickness state on the basis of the first motion sickness state index and the second motion sickness state index.

[19] A motion sickness state determination method including:

a first index acquisition process for acquiring biological information of a subject as a first motion sickness state index;

a second index acquisition process for acquiring a second motion sickness state index on the basis of movement information regarding a movement of the subject and/or surrounding environment information perceived by the subject; and

a motion sickness state determination process for determining whether or not the subject will be in a motion sickness state or is in the motion sickness state on the basis of the first motion sickness state index and the second motion sickness state index.

### REFERENCE SIGNS LIST

1 Motion sickness state determination system
10 Motion sickness state determination device
11 Control unit
12 First index acquisition unit
13 Second index acquisition unit
14 Motion sickness state determination unit
15 Motion sickness state control unit
16 Motion sensor
20 Biological information acquisition device
30 Surrounding environment information acquisition device

1. A motion sickness state determination system comprising:
a first index acquisition unit configured to acquire biological information of a subject as a first motion sickness state index;
a second index acquisition unit configured to acquire a second motion sickness state index on a basis of movement information regarding a movement of the subject and/or surrounding environment information perceived by the subject; and
a motion sickness state determination unit configured to determine whether or not the subject will be in a motion sickness state or is in the motion sickness state on a basis of the first motion sickness state index and the second motion sickness state index.

2. The motion sickness state determination system according to claim 1, further comprising: a motion sickness state control unit configured to control the motion sickness state of the subject on a basis of a determination result of the motion sickness state determination unit.

3. The motion sickness state determination system according to claim 2, wherein the motion sickness state control unit determines processing content used to control the motion sickness state on a basis of information acquired from a database.

4. The motion sickness state determination system according to claim 1, wherein the motion sickness state of the subject is determined in real time.

5. The motion sickness state determination system according to claim 1, wherein in a case where both of the first motion sickness state index and the second motion sickness state index satisfy references set for the respective indexes, the motion sickness state determination unit determines that the subject will be in the motion sickness state or is in the motion sickness state.

6. The motion sickness state determination system according to claim 1, wherein the biological information includes information regarding an enzyme derived from the subject.

7. The motion sickness state determination system according to claim 6, wherein the enzyme includes an amylase.

8. The motion sickness state determination system according to claim 1, wherein
the movement information is movement information regarding a movement of a body of the subject,
the surrounding environment information is video information regarding a video visually perceived by the subject, and
the second index acquisition unit acquires a second motion sickness state index on a basis of the movement information and the video information.

9. The motion sickness state determination system according to claim 8, wherein
the movement information is first acceleration information based on the movement of the body of the subject,
the surrounding environment information is second acceleration information based on a video visually perceived by the subject, and
the second index acquisition unit acquires a second motion sickness state index on a basis of the first acceleration information and the second acceleration information.

10. The motion sickness state determination system according to claim 9, wherein the second index acquisition unit acquires a difference between the first acceleration information and the second acceleration information as the second motion sickness state index.

11. The motion sickness state determination system according to claim 8, further comprising: a motion sickness state control unit configured to apply a current to a head of the subject on a basis of a determination result of the motion sickness state determination unit.

12. The motion sickness state determination system according to claim 1, wherein
the surrounding environment information is odor information regarding an odor perceived by sense of smell by the subject, and
the second index acquisition unit acquires a second motion sickness state index on a basis of the odor information.

13. The motion sickness state determination system according to claim 12, wherein the second index acquisition unit acquires an intensity of a predetermined type of odor as the second motion sickness state index.

14. The motion sickness state determination system according to claim 12, further comprising: a motion sickness state control unit configured to present an odor to the subject or remove an odor around the subject on a basis of a determination result of the motion sickness state determination unit.

15. The motion sickness state determination system according to claim 1, further comprising: an output unit configured to output a determination result of the motion sickness state determination unit.

16. A biological information acquisition device comprising
a biological information acquisition unit configured to acquire biological information of a subject, wherein
the biological information acquisition device is used in combination with an index acquisition device that acquires a motion sickness state index on a basis of movement information regarding a movement of the subject and/or surrounding environment information perceived by the subject in order to determine a motion sickness state of the subject.

17. A surrounding environment information acquisition device comprising:
a surrounding environment information acquisition unit configured to acquire surrounding environment information perceived by a subject, wherein
the surrounding environment information acquisition device is used in combination with a biological information acquisition device that acquires biological information of the subject as a motion sickness state index in order to determine a motion sickness state of the subject.

18. A motion sickness state determination device comprising:
a first index acquisition unit configured to acquire biological information of a subject as a first motion sickness state index;
a second index acquisition unit configured to acquire a second motion sickness state index on a basis of movement information regarding a movement of the subject and/or surrounding environment information perceived by the subject; and
a motion sickness state determination unit configured to determine whether or not the subject will be in a motion sickness state or is in the motion sickness state on a basis of the first motion sickness state index and the second motion sickness state index.

19. A motion sickness state determination method comprising:
a first index acquisition process for acquiring biological information of a subject as a first motion sickness state index;
a second index acquisition process for acquiring a second motion sickness state index on a basis of movement information regarding a movement of the subject and/or surrounding environment information perceived by the subject; and
a motion sickness state determination process for determining whether or not the subject will be in a motion sickness state or is in the motion sickness state on a basis of the first motion sickness state index and the second motion sickness state index.
